(19) United States
(12) Patent Application Publication
(10) Pub. No.: US 2023/0158160 A1
(43) Pub. Date: May 25, 2023

(54) PHARMACEUTICAL COMPOSITION OF EXTENDED-RELEASE ORAL SUSPENSION AND PROCESS FOR PREPARATION THEREOF

(71) Applicants: LYRUS LIFE SCIENCES PVT. LTD., Bangalore (IN); NOKHA TRADING LLP, Bangalore (IN)

(72) Inventors: Chandanmal Pukhraj Bothra, Bangalore (IN); Hemanth Kumar Bothra, Bangalore (IN); Elayaraja Natarajan, Bangalore (IN); Chitra Ajithan Varma, Bangalore (IN); Venkateshappa Govindappa, Bangalore (IN)

(73) Assignees: LYRUS LIFE SCIENCES PVT. LTD., Bangalore (IN); NOKHA TRADING LLP, Bangalore (IN)

(21) Appl. No.: 17/912,424

(22) PCT Filed: May 22, 2021
(86) PCT No.: PCT/IB2021/054459

§ 371 Date: Sep. 16, 2022

(30) Foreign Application Priority Data
May 23, 2020 (IN) 202041021756
May 30, 2020 (IN) 202041022739
Jun. 4, 2020 (IN) 202041023560

(51) Int. Cl. A61K 47/58; A61K 47/36; A61K 31/155; A61K 31/55; A61K 31/445

(52) U.S. Cl. CPC A61K 47/585; A61K 47/36; A61K 31/155; A61K 31/55; A61K 31/445

(57) ABSTRACT

The present invention relates to extended-release suspension composition. The present invention specifically relates to extended-release suspension composition comprising active ingredient and pharmaceutically acceptable excipients, wherein said composition is in the form of powder for suspension or a ready to use suspension. The present invention specifically relates to extended-release suspension composition comprising active ingredient and pharmaceutically acceptable excipients, wherein said composition is devoid of uncoated active ingredient-ion exchange resin complex portion and polyvinyl acetate. The present invention also relates to extended-release suspension composition comprising one or more functional barrier coatings on active ingredient-ion exchange resin complex, wherein said functional barrier coatings comprises hypromellose and/or talc.

### FIELD OF INVENTION

The present invention relates to extended-release suspension composition.

The present invention specifically relates to extended-release suspension composition comprising active ingredient and pharmaceutically acceptable excipients, wherein said composition is in the form of powder for suspension or a ready to use suspension.

The present invention specifically relates to extended-release suspension composition comprising active ingredient and pharmaceutically acceptable excipients, wherein said composition is devoid of uncoated active ingredient-ion exchange resin complex portion and polyvinyl acetate.

The present invention specifically relates to extended-release suspension composition, wherein said composition comprising activated resin and devoid of uncoated active ingredient-ion exchange resin complex portion and polyvinyl acetate.

The present invention more specifically relates to extended-release suspension composition comprising one or more functional barrier coatings on active ingredient-ion exchange resin complex which is devoid of uncoated active ingredient-ion exchange resin complex portion and polyvinyl acetate.

The present invention also relates to extended-release suspension composition comprising one or more functional barrier coatings on active ingredient-ion exchange resin complex, wherein said functional barrier coatings comprise hypromellose and/or talc.

The present invention also relates to extended-release suspension composition prepared by using entrapment technique, where the active ingredient-ion exchange resin complex is entrapped into a polymer, followed by one or more functional barrier coatings.

### BACKGROUND OF INVENTION

Extended release solid compositions can be in the form of tablets or capsules, wherein the release of the active ingredient is controlled by using a reservoir or a matrix system. However, extended release solid compositions suffer from certain drawbacks such as difficulty in swallowing, particularly for certain groups of patients, e.g., pediatrics and geriatrics, resulting in poor patient compliance. Further, high doses of active ingredient lead to large-sized compositions which aggravates this problem.

In view of all this, extended release liquid compositions provide the best alternative over extended release solid compositions. Extended release liquid compositions are easy to administer, thereby leading to enhanced patient compliance. Additionally, extended release liquid compositions provide a unique advantage of having a flexible dosing regimen.

Variety of active ingredients can be incorporated and developed as extended release oral suspension by using this technology which includes Metformin Hydrochloride, Galantamine Hydrobromide, Methylphenidate Hydrochloride, Amphetamine salts and Dextromethorphan Hydrobromide.

U.S. Pat. No. 8,062,667 B2 discloses pharmaceutical preparations comprising active ingredient(s) bound to an ion-exchange resin to provide a active ingredient-ion exchange resin complex, admixing of such complex with a release retardant water-insoluble polymer, and coating of such admixture with a highly flexible, substantially tack-free, non-ionic, water-insoluble, water-permeable diffusion membrane which is preferably aqueous-based and provides a coating membrane that maintains its film integrity, and further provides controllable modified release of the active pharmaceutical(s) in the gastrointestinal tract for a duration of up to about 24 hours.

U.S. Pat. No. 8,287,903 B2 discloses methylphenidate extended release powder blend formulation which is reconstitutable into an orally administered aqueous extended release suspension formulation. The extended release powder blend formulation comprises (i) an immediate release methylphenidate component, (ii) a sustained release barrier coated methylphenidate-ion exchange resin complex-matrix, and (iii) an optional water soluble buffering agent.

All the prior art references discloses extended release oral suspension composition with Immediate release component and with use of polyvinylacetate. The inventors of the present invention have developed extended-release suspension composition which is devoid of uncoated active ingredient-ion exchange resin complex portion and polyvinyl acetate. Further, the composition comprises hypromellose and talc in barrier coatings. The inventors of the present invention have also developed a process for the preparation of extended-release suspension composition.

### OBJECTIVE OF THE INVENTION

The main objective of the present invention is to provide an extended-release suspension composition.

Another objective of the present invention is to provide an extended-release suspension composition comprising active ingredient and pharmaceutically acceptable excipients, wherein said composition is in the form of powder for suspension or a ready to use suspension.

Another objective of the present invention is to provide an extended-release suspension composition comprising active ingredient and pharmaceutically acceptable excipients, wherein said composition is devoid of uncoated active ingredient-ion exchange resin complex portion and polyvinyl acetate.

Another objective of the present invention is to provide an extended-release suspension composition, wherein said composition comprising activated resin and devoid of uncoated active ingredient-ion exchange resin complex portion and polyvinyl acetate.

Another objective of the present invention is to provide an extended-release suspension composition comprising one or more functional barrier coatings on active ingredient-ion exchange resin complex which is devoid of uncoated active ingredient-ion exchange resin complex portion and polyvinyl acetate.

Another objective of the present invention is to provide an extended-release suspension composition comprising one or more functional barrier coatings on active ingredient-ion exchange resin complex, wherein said functional barrier coatings comprise hypromellose and/or talc.

Another objective of the present invention is to provide process for the preparation of extended-release suspension composition.

Another objective of the present invention is to provide an extended-release suspension composition prepared by using entrapment technique, where the active ingredient-ion exchange resin complex is entrapped into a polymer, followed by one or more functional barrier coatings.

### SUMMARY OF THE INVENTION

Accordingly, the present invention relates to an extended-release suspension composition.

Another embodiment of the present invention relates to an extended-release suspension composition comprising active ingredient and pharmaceutically acceptable excipients.

Another embodiment of the present invention relates to an extended-release suspension composition comprising active ingredient and pharmaceutically acceptable excipients, wherein said composition is in the form of powder for suspension or a ready to use suspension.

Another embodiment of the present invention relates to an extended-release suspension composition comprising active ingredient and pharmaceutically acceptable excipients, wherein said composition is devoid of uncoated active ingredient-ion exchange resin complex portion and polyvinyl acetate.

Another embodiment of the present invention relates to an extended-release suspension composition, wherein said composition comprising activated resin and devoid of uncoated active ingredient-ion exchange resin complex portion and polyvinyl acetate.

Another embodiment of the present invention relates to an extended-release suspension composition comprising one or more functional barrier coatings on active ingredient-ion exchange resin complex which is devoid of uncoated active ingredient-ion exchange resin complex portion and polyvinyl acetate.

Another embodiment of the present invention relates to an extended-release suspension composition comprising one or more functional barrier coatings on active ingredient-ion exchange resin complex, wherein said functional barrier coatings comprise hypromellose and/or talc.

Another embodiment of the present invention relates to an extended-release suspension composition comprising;
a) polymeric coated active ingredient-ion exchange resin complex comprising:
   (i) active ingredient particulate matrix comprising active ingredient-ion exchange resin complex,
   (ii) solvation coating on the active ingredient-ion exchange resin complex,
   (iii) one or more functional barrier coating on the pre-coated active ingredient-ion exchange resin complex obtained in step (ii), wherein said functional barrier coating provide modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to an extended-release suspension composition comprising;
a) polymeric coated active ingredient-ion exchange resin complex comprising:
   (i) active ingredient particulate matrix comprising active ingredient-ion exchange resin complex,
   (ii) solvation coating on the active ingredient-ion exchange resin complex,
   (iii) dual release functional barrier coatings on the pre-coated active ingredient-ion exchange resin complex obtained in step (ii), wherein said functional barrier coatings provides dual modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to an extended-release suspension composition comprising;
a) polymeric coated active ingredient-ion exchange resin complex comprising:
   (i) active ingredient particulate matrix comprising active ingredient-ion exchange resin complex,
   (ii) entrapment of active ingredient-ion exchange resin complex in polymer,
   (iii) solvation coating on the active ingredient-ion exchange resin complex obtained in step (ii),
   (iv) dual release functional barrier coatings on the pre-coated active ingredient-ion exchange resin complex obtained in step (iii), wherein said functional barrier coatings provides dual modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to an extended-release suspension composition comprising;
a) polymeric coated active ingredient-ion exchange resin complex comprising:
   (i) active ingredient particulate matrix comprising active ingredient-sodium polystyrene sulfonate complex,
   (ii) solvation coating on the active ingredient-sodium polystyrene sulfonate complex comprising polymers selected from polyethylene glycol, hypromellose and povidone,
   (iii) one or more functional barrier coating on the pre-coated active ingredient-sodium polystyrene sulfonate complex obtained in step (ii), wherein said functional barrier coating comprising hypromellose and talc which provides modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to an extended-release suspension composition comprising;
a) polymeric coated active ingredient-ion exchange resin complex comprising:
   (i) active ingredient particulate matrix comprising active ingredient-sodium polystyrene sulfonate complex,
   (ii) solvation coating on the active ingredient—sodium polystyrene sulfonate complex comprising polymers selected from polyethylene glycol, hypromellose and povidone,
   (iii) dual release functional barrier coatings on the pre-coated active ingredient-sodium polystyrene sulfonate complex obtained in step (ii), wherein said functional barrier coatings comprising hypromellose and talc which provides dual modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to an extended-release suspension composition comprising;
a) polymeric coated active ingredient-ion exchange resin complex comprising:
   (i) active ingredient particulate matrix comprising active ingredient-sodium polystyrene sulfonate complex,
   (ii) entrapment of active ingredient—sodium polystyrene sulfonate complex in ethylcellulose polymer,
   (iii) solvation coating on the active ingredient-sodium polystyrene sulfonate complex comprising polymers selected from polyethylene glycol, hypromellose and povidone,
   (iv) dual release functional barrier coatings on the pre-coated active ingredient-sodium polystyrene sulfonate complex obtained in step (ii), wherein said functional barrier coatings comprising hypromellose and talc which provides dual modified release profile to said pharmaceutically acceptable active ingredient, and
b) pharmaceutically acceptable excipients,
   wherein said composition is devoid of uncoated active ingredient-ion exchange resin complex portion and polyvinyl acetate.

Another embodiment of the present invention relates to a process for the preparation of extended-release suspension composition, wherein said process comprises:
(a) preparing active ingredient-ion exchange resin complex,
(b) coating on active ingredient-ion exchange resin complex with solvation coating polymers,
(c) coating on pre-coated active ingredient-ion exchange resin complex with functional barrier coating polymers, and
(d) mixing of functional coated active ingredient-ion exchange resin complex with excipients.

Another embodiment of the present invention relates to a process for the preparation of extended-release suspension composition, wherein said process comprises;
(a) preparing active ingredient-ion exchange resin complex,
(b) coating on active ingredient-ion exchange resin complex with solvation coating polymers,
(c) coating on pre-coated active ingredient-ion exchange resin complex with functional barrier coating polymers,
(d) preparing the placebo granules with excipients,
(e) mixing of functional coated active ingredient-ion exchange resin complex particles obtained in steps (c) and placebo granules obtained in step (d) with pharmaceutically acceptable excipients, and
(f) packing the composition obtained in step (e) in suitable container.

Another embodiment of the present invention relates to a process for the preparation of extended-release suspension composition, wherein said process comprises;
(a) preparing active ingredient—ion exchange resin complex,
(b) coating on active ingredient-ion exchange resin complex with solvation coating polymers,
(c) coating 70% to 95% of pre-coated active ingredient-ion exchange resin complex particles obtained in step (b) with functional barrier coating polymers from 25% w/w to 60% w/w build up,
(d) coating 5% to 30% of pre-coated active ingredient-ion exchange resin complex particles obtained in step (b) with functional barrier coating polymers from 5% w/w to 15% w/w build up,
(e) mixing of functional coated active ingredient-ion exchange resin complex particles obtained in steps (c) and step (d) with pharmaceutically acceptable excipients, and
(f) packing the composition obtained in step (e) in suitable container.

Another embodiment of the present invention relates to a process for the preparation of extended-release suspension composition, wherein said process comprises;
(a) preparing active ingredient—ion exchange resin complex,
(b) coating on active ingredient-ion exchange resin complex with solvation coating polymers,
(c) entrapping the active ingredient-ion exchange resin complex obtained in step (b) with polymers,
(d) coating 70% to 95% of pre-coated active ingredient-ion exchange resin complex particles obtained in step (c) with functional barrier coating polymers from 25% w/w to 60% w/w build up,
(e) coating 5% to 30% of pre-coated active ingredient-ion exchange resin complex particles obtained in step (c) with functional barrier coating polymers from 5% w/w to 15% w/w build up,
(f) mixing of functional coated active ingredient-ion exchange resin complex particles obtained in steps (d) and step (e) with pharmaceutically acceptable excipients, and
(g) packing the composition obtained in step (f) in suitable container.

Another embodiment of the present invention relates to a process for the preparation of extended-release suspension composition, wherein said process comprises;
(a) preparing active ingredient-ion exchange resin complex by adding active ingredient and activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating pre-coated active ingredient-ion exchange resin complex particles obtained in step (b) with functional barrier coating solution comprising hypromellose and talc,
(d) mixing of functional coated active ingredient-ion exchange resin complex particles obtained in steps (c) with pharmaceutically acceptable excipients, and
(e) packing the composition obtained in step (d) in suitable container.

Another embodiment of the present invention relates to a process for the preparation of extended-release suspension composition, wherein said process comprises;
(a) preparing active ingredient-ion exchange resin complex by adding active ingredient and activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating pre-coated active ingredient-ion exchange resin complex particles obtained in step (b) with functional barrier coating solution comprising hypromellose and talc,
(d) preparing placebo granules with excipients,
(e) mixing of functional coated active ingredient-ion exchange resin complex particles obtained in steps (c) with placebo granules obtained in step (d) and pharmaceutically acceptable excipients, and
(f) packing the composition obtained in step (e) in suitable container.

Another embodiment of the present invention relates to a process for the preparation of extended-release suspension composition, wherein said process comprises;
(a) preparing active ingredient-ion exchange resin complex by adding active ingredient, activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating 70% to 95% of pre-coated active ingredient-ion exchange resin complex particles obtained in step (c) with functional barrier coating solution comprising hypromellose and talc from 25% w/w to 60% w/w build up,
(d) coating 5% to 30% of pre-coated active ingredient-ion exchange resin complex particles obtained in step (c) with functional barrier coating solution comprising hypromellose and talc from 5% w/w to 15% w/w build up,
(e) mixing of functional coated active ingredient-resin complex particles obtained in steps (c) and (d) with pharmaceutically acceptable excipients, and
(f) packing the composition obtained in step (e) in suitable container.

Another embodiment of the present invention relates to a process for the preparation of extended-release suspension composition, wherein said process comprises;
(a) preparing active ingredient-ion exchange resin complex by adding active ingredient, activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating 70% to 95% of pre-coated active ingredient-ion exchange resin complex particles obtained in step (c) with functional barrier coating solution comprising hypromellose and talc from 25% w/w to 60% w/w build up,
(d) coating 5% to 30% of pre-coated active ingredient-ion exchange resin complex particles obtained in step (c) with functional barrier coating solution comprising hypromellose and talc from 5% w/w to 15% w/w build up,
(e) preparing placebo granules with excipients,
(f) mixing of functional coated active ingredient-resin complex particles obtained in steps (c) and (d) with placebo granules obtained in step (e) and pharmaceutically acceptable excipients, and
(g) packing the composition obtained in step (f) in suitable container.

Another embodiment of the present invention relates to a process for the preparation of extended-release suspension composition, wherein said process comprises;
(a) preparing active ingredient-ion exchange resin complex by adding active ingredient, activated resin to solvent under stirring for 7-8 hours followed by drying,
(b) entrapping the active ingredient-ion exchange resin complex obtained in step (a) using ethylcellulose solution,
(c) coating the active ingredient-ion exchange resin complex obtained in step (b) with solvation coating solution comprising polymer selected from polyethylene glycol, hypromellose and povidone,
(d) coating 70% to 95% of pre-coated active ingredient-ion exchange resin complex particles obtained in step (d) with functional barrier coating solution comprising hypromellose and talc from 25% w/w to 60% w/w build up,
(e) coating 5% to 30% of pre-coated active ingredient-ion exchange resin complex particles obtained in step (d) with functional barrier coating solution comprising hypromellose and talc from 5% w/w to 15% w/w build up,
(f) mixing of functional coated active ingredient-resin complex particles obtained in steps (d) and (e) with pharmaceutically acceptable excipients, and
(g) packing the composition obtained in step (f) in suitable container.

Another embodiment of the present invention relates to a process for the preparation of extended-release suspension composition, wherein said process comprises;
(a) preparing active ingredient-ion exchange resin complex by adding active ingredient, activated resin to solvent under stirring for 7-8 hours followed by drying,
(b) entrapping the active ingredient-ion exchange resin complex obtained in step (a) using ethylcellulose solution,
(c) coating the active ingredient-ion exchange resin complex obtained in step (b) with solvation coating solution comprising polymer selected from polyethylene glycol, hypromellose and povidone,
(d) coating 70% to 95% of pre-coated active ingredient-ion exchange resin complex particles obtained in step (d) with functional barrier coating solution comprising hypromellose and talc from 25% w/w to 60% w/w build up,
(e) coating 5% to 30% of pre-coated active ingredient-ion exchange resin complex particles obtained in step (d) with functional barrier coating solution comprising hypromellose and talc from 5% w/w to 15% w/w build up,
(f) preparing the placebo granules with excipients,
(g) mixing of functional coated active ingredient-resin complex particles obtained in steps (d) and (e) with placebo granules obtained in step (f) and pharmaceutically acceptable excipients, and
(h) packing the composition obtained in step (g) in suitable container.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

In still yet another embodiment of the present invention, the pharmaceutical composition of the invention also relates to a wide variety of active pharmaceutical ingredient's suitable for use in extended release formulations. Representative active pharmaceutical ingredient's may include antibacterial, antacids, analgesic and anti-inflammatory agents, anti-arrhythmic agents, antiprotozoal agents, anti-coagulants, antidepressants, anti-diabetic agents, anti-epileptic agents, antifungal agents, antihistamines, anti-hypertensive agents, anti-muscarinic agents, antineoplastic agents, antimetabolites, anti-migraine agents, anti-Parkinsonian agents, antipsychotic, hypnotic and sedating agents, anti-stroke agents, antitussive, antivirals, cardiac inotropic agents, corticosteroids, disinfectants, diuretics, enzymes, essential oils, gastro-intestinal agents, haemostatics, lipid regulating agents, local anaesthetics, opioid analgesics, parasympathomimetics and anti-dementia drugs, peptides and proteins, sex hormones, stimulating agents, vasodilators or mixtures thereof.

According to an embodiment of the present invention, active pharmaceutical ingredient is selected from the group consisting of Acebutolol hydrochloride, Amantadine hydrochloride, Amodiaquine hydrochloride, Amphetamine salts, Chloroquine phosphate, Chloroquine sulphate, Chlorpromazine hydrochloride, Promethazine Hydrochloride, Ciprofloxacin hydrochloride, Cloxacillin sodium, Dextroamphetamine salts, Diltiazem hydrochloride, Doxycycline hydrochloride, Galantamine Hydrobromide, Levamisole hydrochloride, Lincomycin hydrochloride, Fexofenadine Hydrochloride, Cetirizine Hydrochloride, Diphenhydramine Hydrochloride, Bromhexine Hydrochloride, Dextromethorphan Hydrobromide, Tramadol Hydrochloride, Mebeverine hydrochloride, Metformin hydrochloride, Methylphenidate Hydrochloride, Sertraline Hydrochloride, Donepezil Hydrochloride, Fluoxetine Hydrochloride, Trifluoperazine Hydrochloride, Promazine Hydrochloride, Oxybutynin Hydrochloride, Oxprenolol hydrochloride, Oxytetracycline hydrochloride, Procainamide hydrochloride, Pseudoephedrine hydrochloride, Quinine bisulphate, Ranitidine hydrochloride, Streptomycin sulphate, Tetracycline hydrochloride, potassium chloride, Verapamil hydrochloride and the like and their pharmaceutically acceptable salts, ester and hydrates.

Specifically the active ingredients include Metformin Hydrochloride, Galantamine Hydrobromide, Methylphenidate Hydrochloride, Amphetamine salts and Dextromethorphan Hydrobromide.

The concentration of active pharmaceutical ingredient used in the extended release suspension composition is from 0.5% to 50% (w/w) of total weight of the composition.

Active ingredient-ion exchange resin complex is formed in such a manner that Active Ingredient is bound to a pharmaceutically acceptable water insoluble ion exchange resin to form said active ingredient-ion exchange resin complex.

The ion exchange resin as used herein is being selected from
(A) a sulfonated copolymer comprising styrene and divinylbenzene, and
(B) a copolymer comprising styrene and divinylbenzene having quaternary ammonium functional groups.

Ion exchange resin used in the present invention is sodium polystyrene sulfonate.

The concentration of ion exchange resin in the extended release suspension composition is from 2% to 50% (w/w) of total weight of the composition.

Active ingredient-ion exchange resin complex wherein the active ingredient and resin ratio is from 1:1 to 1:3.

Chelating agent used in active ingredient resin complex is to enhance the complexation process and stability to the product. The chelating agent as used herein is selected from EDTA, a salt of EDTA, diaminocyclohexanetetraacetic acid (DCTA), diethylenetriaminepentaacetic acid, bis(aminoethyl)glycolether-N,N,N′,N′-tetraacetic acid, iminodiacetic acid or a salt thereof. More preferably, the chelating agent is selected from EDTA or a salt of EDTA.

The concentration of chelating agent in the extended release suspension composition is from 0.02% to 5% (w/w) of total weight of the composition.

Solvation coating over active ingredient-ion exchange resin complex is to enable the effective application thereto of diffusion barrier coatings, resulting in the ability to effectively prolong the release of active ingredients from active ingredient-ion exchange resin complexes. The solvation coating agent as used in the present invention is selected from the group consisting of polyethylene glycol, mannitol, hypromellose, hydroxy propyl cellulose, povidone and methylcellulose. Preferably, the solvation coating agent is hypromellose, Polyethylene glycol, and povidone.

The concentration of solvation coating agent in the extended release suspension composition is from 0.1% to 10% (w/w) of total weight of the composition.

The entrapment polymers as used in the present invention are selected from the group consisting of a cellulose-derived material such as ethyl cellulose, methyl cellulose, carboxymethyl cellulose, hydroxypropylmethyl cellulose, Methacrylic acid polymers & its co-poymers, sodium alginates, sodium carboxymethyl cellulose. Preferably, entrapment polymer is ethyl cellulose.

The concentration of entrapment polymers in the extended release suspension composition is from 0.1% to 10% (w/w) of total weight of the composition.

The polymers used for the one or more functional barrier coatings on the active ingredient-ion exchange resin complex as used herein may be water soluble polymers, water insoluble polymers, water permeable polymers.

Water soluble polymers used alone or in combination in the compositions of the present invention include, but are not limited to gum arabic or a partially or totally synthetic polymer selected from the alkyl celluloses and derivatives thereof such as methylcellulose, ethylcellulose, hydroxyalkyl celluloses and derivatives thereof such as hydroxyethyl celluloses, hydroxypropyl celluloses, hydroxyalkyl alkylcelluloses and derivatives thereof such as the hydroxypropylmethyl celluloses (hypromelloses), carboxyalkylcelluloses such as carboxymethylcelluloses, polyvinylpyrrolidones, copolymers of N-vinylpyrrolidone and vinyl acetate or combinations of said polymers and derivatives and mixtures thereof. Preferably used functional barrier coating polymers are ethyl cellulose and hypromellose.

Water insoluble polymers used alone or in combination in the compositions of the present invention include, but are not limited to acrylic resin, polymers or coplymers of acrylic acid, methyl acrylate, ethyl acrylate, methacrylic acid, methyl methacrylate, ethyl methacrylate and the like which may contain quaternary ammonium groups such as ammonio (meth)acrylate copolymers. Preferred examples are copolymers of ethyl acrylate, methyl methacrylate and trimethylammonioethyl methacrylate chloride. Such an acrylic polymer is available under the name Eudragit RS which is a water-insoluble copolymer (poly(ethyl acrylate, methyl methacrylate, trimethylammonioethyl methacrylate chloride) 1:2:0.1, manufactured by Rh6m Pharma, Germany) e.g. in form of organic-based polymeric solutions or aqueous-based polymeric dispersions thereof which may be used for coating, for example Eudragit RS 30D. Another acrylic polymer may be Eudragit RL which consists of the same components as Eudragit RS but has a different molar ratio (Eudragit RL: poly(ethylacrylate, methyl methacrylate, trimethylammonioethyl methacrylate chloride; 1:2:0.2) e.g. in form of organic-based polymeric solutions or aqueous-based polymeric dispersions thereof, for example Eudragit RL 30D.

Water permeable polymers used alone or in combination in the compositions of the present invention include, but are not limited to non-acrylic polyolefins, such as low density polyethylene, high density polyethylene, polypropylene, polystyrene, polyvinylchloride, acrylonitrilebutadiene-styrene terpolymers, styrene-acrylonitrile copolymer, styrene butadiene copolymers, poly (4-methyl-pentene-1), polybutylene, polyvinylidene chloride, polyvinyl butyral, chlorinated polyethylene, ethylene-vinyl acetate copolymers, polyvinyl acetate, polyvinyl alcohol, and combinations and blends thereof; acrylic polyoflefins, such as polymethyl-methacrylate, polymethyl-acrylate, ethyleneacrylic acid copolymers, ethylene-acrylic acid metal salt copolymers, and combinations and blends thereof; oxidation polymers, including polyphenylene oxide; and condensation polymers including polyethylene terephthalate, polybutylene terephthalate, Nylon 6, Nylon 11, Nylon 13, Nylon-6,6, polycarbonates, polysulfone, and combinations and blends thereof.

The concentration of barrier coating polymers in the extended release suspension composition is from 0.1% to 10% (w/w) of total weight of the composition.

The compositions of the present invention may further include plasticizer, taste masking agents, sweetening agent, buffering agents, anti-tacking agents, suspending agents/thickeners, preservatives, flavouring agents, surfactants, and lubricants.

Plasticizer is used alone or in combination in the compositions of the present invention include, but are not limited to dibutyl sebacate, propylene glycol, polyethylene glycol, polyvinyl alcohol, triethyl citrate, acetyl triethyl citrate, acetyl tributyl citrate, tributyl citrate, triacetin, and Soluphor® P (2-pyrrolidone), and mixtures thereof. Preferably, the plasticizer is dibutyl sebacate.

The concentration of plasticizer in the extended release suspension composition is from 0.01% to 10% (w/w) of total weight of the composition.

Taste masking agents used alone or in combination in the compositions of the present invention include, but are not limited to osmolality adjusting ingredient, flavouring agent and film-forming agents. Osmolality adjusting ingredient is selected from potassium chloride, calcium chloride, sodium lactate, sodium chloride, dextrose, mannitol, sucrose, trehalose, and phosphate buffered saline, or mixtures thereof. Flavouring agent is selected from vanillin, ethyl vanillin, menthol, citric acid, fumaric acid ethyl maltitol, and tartaric acid. Film-forming agent is selected from pullulan, hydroxypropylmethyl cellulose, hydroxyethyl cellulose, hydroxypropyl cellulose, polyvinyl pyrrolidone, carboxymethyl cellulose, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

The concentration of taste masking agents used in the present compositions is in the range of 0.05% to 20% (w/w) of total weight of the composition.

Sweetening agents used alone or in combination in the compositions of the present invention include, but are not limited to saccharin, saccharin sodium, aspartame, maltitol, zinc gluconate, ethyl maltitol, glycine, acesulfame-K, honey, golden syrup, misri, spray dried licorice root, glycerrhizin, dextrose, sodium gluconate, stevia powder; glucono delta-lactone, ethyl vanillin, vanillin, sucrose, glucose (corn syrup), dextrose, invert sugar, fructose, high fructose corn syrup, sucralose, stevia, dihydrochalcone, maltodextrin, polydextrose, sugar alcohols such as sorbitol, sorbitol syrup, mannitol, xylitol, hexa-resorcinol. Preferably used sweetening agent is sucrose, sucralose, xylitol, high fructose corn syrup and combinations thereof.

The concentration of sweetening agent used in the present compositions is in the range of 0.1% to 90% (w/w) of total weight of the composition.

pH adjusting agents/buffering agent used alone or in combination in the compositions of the present invention include, but are not limited to phosphoric acid, monobasic sodium or potassium phosphate, triethanolamine (TRIS), BICINE, HEPES, Trizma, glycine, histidine, arginine, lysine, asparagine, aspartic acid, glutamine, glutamic acid, carbonate, bicarbonate, potassium metaphosphate, potassium phosphate, monobasic sodium acetate, acetic acid, acetate, citric acid, sodium citrate anhydrous, sodium citrate dihydrate and combinations thereof. In certain embodiments, an acid or a base is added to adjust the pH. Suitable acids or bases include HCL, NaOH and KOH. Preferably used pH adjusting agent is citric acid, sodium citrate anhydrous, sodium citrate dihydrate.

The concentration of pH adjusting agents used in the present compositions is in the range of 0.01% to 10% (w/w) of total weight of the composition.

Anti-tacking agents used alone or in combination in the compositions of the present invention include, but are not limited to silicon dioxide, titanium dioxide, alumina, talc, kaolin, powdered cellulose and microcrystalline cellulose. Preferably, anti-tacking agents used in the composition are silicon dioxide, talc.

The concentration of anti-tacking agents used in the present compositions is in the range of 0.01% to 5% (w/w) of total weight of the composition.

Suspending agents/thickeners, used alone or in combination in the compositions of the present invention include, but are not limited to xanthan gum, microcrystalline cellulose, starch, acacia, hydroxypropyl cellulose, hydroxypropyl methylcellulose, carboxymethyl cellulose, sodium carboxymethyl cellulose, tragacanth and combinations thereof. Preferably, Suspending agents/thickeners used are xanthan gum, microcrystalline cellulose, starch, carboxymethyl cellulose, sodium carboxymethyl cellulose and combinations thereof.

The concentration of Suspending agents/thickeners used in the present compositions is in the range of 0.01% to 20% (w/w) of total weight of the composition.

Preservatives used alone or in combination in the compositions of the present invention include, but are not limited to methylparaben, propylparaben, sodium methylparaben, sodium propylparaben, Sodium metabisulphite, sodium benzoate, benzoic acid, disodium EDTA, citric acid, tartaric acid, fumaric acid, malic acid and mixtures thereof. Preferably, preservatives are methylparaben, propylparaben, sodium methylparaben, sodium propylparaben, Sodium metabisulphite and combinations thereof.

The concentration of preservatives used in the present compositions is in the range of 0.01% to 10% (w/w) of total weight of the composition.

Flavouring agents used alone or in combination in the compositions of the present invention include, but are not limited to coffee extract, mint, lamiacea extracts, lemon flavour, almond oil, babassu oil, banana flavour, flavour orange, bubblegum flavour, borage oil, blackcurrant seed oil, canola oil, castor oil, coconut oil, corn oil, cottonseed oil, evening primrose oil, grape seed oil, strawberry flavor, groundnut oil, mustard seed oil, olive oil, palm oil, palm kernel oil, peanut oil, grape seed oil, safflower oil, sesame oil, shark liver oil, soybean oil, sunflower oil; hydrogenated castor oil, hydrogenated coconut oil, hydrogenated palm oil, hydrogenated soybean oil, hydrogenated vegetable oil, hydrogenated cottonseed and castor oil, partially hydrogenated soybean oil, soy oil, glyceryl tricaproate, glyceryl tricaprylate, glyceryl tricaprate, glyceryl triundecanoate, glyceryl trilaurate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, saturated polyglycolized glycerides, linoleic glycerides, caprylic/capric glycerides, modified triglycerides, fractionated triglycerides, safrole, citric acid, d-limonene, malic acid and phosphoric acid or salts and/or mixtures thereof. Preferably used flavouring agent is strawberry flavour, banana flavour, flavour orange, bubblegum flavour.

The concentration of flavouring agent used in the compositions is in the range of 0.1% to 20% (w/w) of total weight of the composition.

Surfactants used alone or in combination in the compositions of the present invention include, but are not limited to SLS, poloxamers (e.g., poloxamer 188), glycerol Anhydrous, polysorbate (e.g., 20 or 80), stearyl hetanoate, caprylic/capric fatty acid esters of saturated fatty alcohols of chain length C12-C-18, isostearyl diglycerol isostearic acid, sodium dodecyl sulphate, isopropyl myristate, isopropyl palmitate, and isopropyl myristate/isopropyl stearate/isopropyl palmitate mixture. Preferably surfactants are poloxamers (e.g., poloxamer 188), glycerol Anhydrous, polysorbate.

The concentration of surfactant used in the compositions is in the range of 0.01% to 20% (w/w) of total weight of the composition.

Lubricants used alone or in combination in the compositions of the present invention include, but are not limited to magnesium stearate, stearic acid, sodium stearyl fumarate, microcrystalline cellulose, cro sslinked sodium carboxymethylcellulose, silica, aerosil, corn starch, sucrose fatty acid esters, polyethylene glycol, talc, stearic acid, calcium stearate.

The concentration of lubricants used in the present compositions with increased bio absorption is in the range of 1% to 5% w/w of total weight of the composition.

Suitable solvents used in compositions of the present invention selected from the group comprising water, ethanol, methylene chloride, isopropyl alcohol, acetone, methanol, or combinations thereof.

Another embodiment of the present invention relates to an extended-release suspension composition comprising functional coated active ingredient-ion exchange resin complex, placebo granules, and pharmaceutically acceptable excipients.

Another embodiment of the present invention relates to a process for the preparation of extended-release suspension composition, wherein said process comprises packing the functional coated active ingredient-ion exchange resin complex particles with pharmaceutically acceptable excipients and optionally with placebo granules in a suitable container.

Another embodiment of the present invention relates to extended-release suspension composition, wherein said composition is devoid of uncoated active ingredient-ion exchange resin complex portion.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### Example 1: Ready to Use Suspension

### Formula for Active Ingredient-Resin Complexation

| | | |
|---|---|---|
| S. No. | Ingredients | mg/ml |

Step 1 active ingredient-resin complexation

| | | |
|---|---|---|
| 1 | Amphetamine (EQ to Amphetamine base) | 7.50 |
| 2 | sodium polystyrene sulfonate | 22.50 |
| 3 | disodium EDTA | 2.00 |
| 4 | purified water | Q.S |

| | |
|---|---|
| Total weight active ingredient-resin complex | 32.00 |

Step 2 Solvation coating (Pre-coating/solvation coating
of active ingredient-resin complex) - Build up: 20% w/w

| | | |
|---|---|---|
| 5 | polyethylene glycol (PEG) | 6.40 |
| 6 | isopropyl alcohol and purified water | Q.S |

| | |
|---|---|
| Total weight of pre-coated active ingredient-resin complex | 38.4 |

Step 3a Functional coating on pre-coated active ingredient-resin complex
(sustained release coating1) - 50% w/w build up on 80 parts of
pre-coated active ingredient-resin complex

| | | |
|---|---|---|
| 7 | ethylcellulose 10 cps | 11.06 |
| 8 | hypromellose | 1.23 |
| 9 | dibutyl sebacate | 1.54 |
| 10 | talc | 1.54 |
| 11 | isopropyl alcohol and purified water | Q.S |

| | |
|---|---|
| Total weight of functional coated granules (a) | 46.09 |

Step 3b Functional coating on pre-coated active ingredient-resin complex
(sustained release 2 coating) - 5% w/w build up on 20 parts of
pre-coated active ingredient-resin complex

| | | |
|---|---|---|
| 12 | ethylcellulose 10 cps | 0.28 |
| 13 | hypromellose | 0.03 |
| 14 | dibutyl sebacate | 0.04 |
| 15 | talc | 0.04 |
| 16 | isopropyl alcohol and purified water | Q.S |

| | |
|---|---|
| Total weight of functional coated granules (b) | 8.07 |

### Formula for Oral Extended Release Suspension

| S. No | Ingredients | mg/ml |
|---|---|---|
| 1 | functional coated granules (sustained release coated | 46.09 |
| | particulates a - 50% w/w) | |
| 2 | functional coated granules (sustained release coated | 8.07 |
| | particulates b - 5% w/w) | |
| 3 | citric acid anhydrous | 1.70 |
| 4 | propylene glycol | 40.00 |
| 5 | methyl paraben | 1.00 |
| 6 | propyl paraben | 0.10 |
| 7 | xanthan gum | 1.00 |
| 8 | sucralose | 6.00 |
| 9 | bubblegum flavour | 1.00 |
| 10 | Avicel RC 591 | 12.50 |
| 11 | purified water | q.s. |

| | |
|---|---|
| Total weight of oral extended release suspension | 117.46 |

### Manufacturing Process

Step 1: (Active Ingredient-Resin Complex Preparation)

The resin is activated by soaking in water for 1-2 hours. The active ingredient-resin complex was prepared by adding Amphetamine (eq to amphetamine base), sodium polystyrene sulfonate and disodium EDTA to purified water and stirred for 7-8 hours. After complexation, the active ingredient-resin dispersion was kept still for active ingredient-resin complex to settle down and the solvent was drained. The wet drug-resin complex was collected by filtration and dried using fluidized bed dryer.

Step 2: (Pre-Coating of Active Ingredient-Resin Complex)

The coating solution was prepared by dispersing polyethylene glycol in isopropyl alcohol and purified water under stirring for 45 minutes. The coating process was performed in a fluid bed processor by applying coating solution to the active ingredient-resin complex.

Step 3a: (Functional Coating on Pre-Coated Active Ingredient-Resin Complex (Sustained Releasecoating 1)-50% w/w Build Up on 80 Parts of Pre-Coated Active Ingredient-Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 4: (Preparation of Suspension)

Functional coated granules (50% w/w) and Functional coated granules (5% w/w) were mixed with citric acid anhydrous, propylene glycol, methyl paraben, propyl paraben, xanthan gum, sucralose, bubblegum flavour, Avicel RC 591 and water. the suspension was packed in suitable container.

### Example 2: Powder for Suspension

### Formula for Active Ingredient-Ion Exchange Resin Complexation

| | | |
|---|---|---|
| S. No. | Ingredients | mg/ml |

Step 1 Active ingredient-resin complexation

| | | |
|---|---|---|
| 1 | Methylphenidate HCl | 5.00 |
| 2 | sodium polystyrene sulfonate | 15.00 |
| 3 | purified water | Q.S |

| | |
|---|---|
| Total weight of active ingredient- resin complex | 20.00 |

Step 2 Solvation coating (pre-coating of active ingredient-resin complex),
buildup: 20% w/w

| | | |
|---|---|---|
| 4 | polyethylene glycol (PEG) | 4.00 |
| 5 | isopropyl alcohol and purified water | Q.S |

| | |
|---|---|
| Total weight of pre-coated active ingredient-resin complex | 24.00 |

Step 3a Functional coating on pre-coated active ingredient-resin complex
(sustained release 1 coating) - 50% w/w build up on 80 parts of
pre-coated active ingredient- resin complex

| | | |
|---|---|---|
| 6 | ethylcellulose | 6.912 |
| 7 | hypromellose | 0.691 |
| 8 | dibutyl sebacate | 0.960 |
| 9 | talc | 0.960 |
| 10 | isopropyl alcohol and purified water | Q.S |

| | |
|---|---|
| Total weight of functional coated granules (a) | 28.723 |

Step 3b Functional coating on pre-coated active ingredient-resin complex
(sustained release 2 coating) - 5% w/w build up on 20 parts of
pre-coated active ingredient-resin complex

| | | |
|---|---|---|
| 11 | ethylcellulose | 0.173 |
| 12 | hypromellose | 0.019 |
| 13 | dibutyl sebacate | 0.024 |
| 14 | talc | 0.024 |
| 15 | isopropyl alcohol and purified water | Q.S |

| | |
|---|---|
| Total weight of functional coated granules (b) | 5.040 |

### Formula for Placebo Granules

| S. No. | Ingredients | mg/ml |
|---|---|---|
| 1 | poloxamer 188 | 0.72 |
| 2 | sucrose | 200.10 |
| 3 | sodium citrate | 5.60 |
| 4 | anhydrous citric acid | 7.49 |
| 5 | sodium benzoate | 2.89 |
| 6 | purified water | Q.S |

| | |
|---|---|
| Total weight of placebo granules (c) | 216.8 |

### Formula for Powder for Oral Suspension

| S. No. | Ingredients | mg/ml |
|---|---|---|
| 1 | placebo granules (c) | 216.8 |
| 2 | functional coated granules (a) | 28.723 |
| 3 | functional coated granules (b) | 5.040 |
| 4 | starch | 24.55 |
| 5 | xanthan gum | 2.64 |
| 6 | banana flavour | 1.00 |
| 7 | talc | 2.12 |

| | |
|---|---|
| Total weight of composition | 280.87 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Methylphenidate HCl and sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating Solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—50% w/w Build Up on 80 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4: (Preparation of Placebo Granules)

Placebo granules were prepared by mixing poloxamer 188, sucrose, sodium citrate, anhydrous citric acid and sodium benzoate in granulator. granules obtained were dried.

Step 5: (Preparation of Powder for Suspension)

Functional coated granules (50% w/w), functional coated granules (5% w/w) and placebo granules were mixed with starch, xanthan gum, banana flavour and talc. the final blend was packed in suitable container.

### Example 3: Powder for Suspension

### Formula for Active Ingredient-Ion Exchange Resin Complexation

| | | |
|---|---|---|
| S. No. | Ingredients | mg/ml |

Step 1 Active ingredient-ion exchange resin complexation

| | | |
|---|---|---|
| 1 | Methylphenidate HCl | 5.00 |
| 2 | sodium polystyrene sulfonate | 15.00 |
| 3 | purified water | Q.S |

| | |
|---|---|
| Total weight of active ingredient- resin complex | 20.00 |

Step 2 Solvation coating (Pre-coating of
active ingredient-resin complex), Buildup: 20% w/w

| | | |
|---|---|---|
| 4 | polyethylene glycol (PEG) | 4.00 |
| 5 | isopropyl alcohol and purified water | Q.S |

| | |
|---|---|
| Total weight of pre-coated active ingredient-resin complex | 24.00 |

Step 3a Functional coating on pre-coated active ingredient-resin complex
(sustained release 1 coating) - 50% w/w build up on 80 parts of
pre-coated active ingredient-ion exchange resin complex

| | | |
|---|---|---|
| 6 | ethylcellulose | 6.912 |
| 7 | hypromellose | 0.691 |
| 8 | dibutyl sebacate | 0.960 |
| 9 | talc | 0.960 |
| 10 | isopropyl alcohol and purified water | Q.S |

| | |
|---|---|
| Total weight of functional coated granules (a) | 28.723 |

Step 3b Functional coating on pre-coated active ingredient-resin complex
(sustained release 2 coating) - 5% w/w build up on 20 parts of
pre-coated active ingredient-ion exchange resin complex

| | | |
|---|---|---|
| 11 | ethylcellulose | 0.173 |
| 12 | hypromellose | 0.019 |
| 13 | dibutyl sebacate | 0.024 |
| 14 | talc | 0.024 |
| 15 | isopropyl alcohol and purified water | Q.S |

| | |
|---|---|
| Total weight of functional coated granules b) | 5.040 |

### Formula for Placebo Granules

| S. No. | Ingredients | mg/ml |
|---|---|---|
| 1 | poloxamer 188 | 0.72 |
| 2 | sucrose | 200.10 |
| 3 | sodium citrate | 11.40 |
| 4 | anhydrous citric acid | 1.65 |
| 5 | sodium methyl paraben | 0.39 |
| 6 | sodium propyl paraben | 0.04 |
| 7 | sucralose | 1.15 |
| 8 | purified water | Q.S |

| | |
|---|---|
| Total weight of placebo granules (c) | 215.5 |

### Formula for Powder for Oral Suspension

| S. No. | Ingredients | mg/ml |
|---|---|---|
| 1 | placebo granules (c) | 215.5 |
| 2 | functional coated granules (a) | 28.723 |
| 3 | functional coated granules (b) | 5.040 |
| 4 | starch | 24.55 |
| 5 | xanthan gum | 2.64 |
| 6 | banana flavour | 1.00 |
| 7 | silicon dioxide | 1.00 |
| 8 | talc | 2.12 |

| | |
|---|---|
| Total weight of composition | 280.5 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Methylphenidate HCl and Sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating solution was prepared by dispersing polyethylene glycol (PEG) in isopropyl alcohol and purified water under stirring for 45 minutes. the coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—50% w/w Build Up on 80 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. the coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. the coating process was performed in a fluid bed processor by applying coating solution to the pre-coated active ingredient-resin complex.

Step 4: (Preparation of Placebo Granules)

Placebo granules were prepared by mixing poloxamer 188, sucrose, sodium citrate, anhydrous citric acid sucralose, sodium methyl paraben and sodium propyl paraben in granulator. Granules obtained were dried.

Step 5: (Preparation of Powder for Suspension)

Functional coated granules (50% w/w), functional coated granules (5% w/w) and placebo granules were mixed with starch, xanthan gum, banana flavour, silicon dioxide and talc. The final blend was packed in suitable container.

### Example 4: Powder for Suspension

### Formula for Active Ingredient-Ion Exchange Resin Complexation

| | | |
|---|---|---|
| S. No. | Ingredients | mg/ml |

Step 1 Active ingredient-ion exchange resin complexation

| | | |
|---|---|---|
| 1 | Methylphenidate HCl | 5.00 |
| 2 | sodium polystyrene sulfonate | 15.00 |
| 3 | purified water | Q.S |

| | |
|---|---|
| Total weigh of active ingredient-resin complex | 20.00 |

Step 2 Solvation coating (pre-coating of active ingredient-resin complex),
buildup: 20% w/w

| | | |
|---|---|---|
| 4 | polyethylene glycol (PEG) | 4.00 |
| 5 | isopropyl alcohol and purified water | Q.S |

| | |
|---|---|
| Total weight of pre-coated active ingredient- resin complex | 24.00 |

Step 3 Functional coating on pre-coated active ingredient-resin complex
(sustained release coating) - 35% w/w build up on pre-coated
active ingredient-ion exchange resin complex

| | | |
|---|---|---|
| 6 | ethylcellulose | 4.70 |
| 7 | hypromellose | 2.02 |
| 8 | dibutyl sebacate | 0.84 |
| 9 | talc | 0.84 |
| 10 | isopropyl alcohol and purified water | Q.S |

| | |
|---|---|
| Total weight of functional coated granules | 16.8 |

### Formula for Placebo Granules

| S. No. | Ingredients | mg/ml |
|---|---|---|
| 1 | poloxamer 188 | 0.72 |
| 2 | sucrose | 200.10 |
| 3 | sodium citrate | 11.40 |
| 4 | anhydrous citric acid | 1.65 |
| 5 | sodium methyl paraben | 0.39 |
| 6 | sodium propyl paraben | 0.04 |
| 7 | sucralose | 1.15 |
| 8 | purified water | Q.S |

| | |
|---|---|
| Total weight of placebo granules | 215.5 |

### Formula for Powder for Oral Suspension

| S. No. | Ingredients | mg/ml |
|---|---|---|
| 1 | placebo granules | 215.5 |
| 2 | functional coated granules | 16.80 |
| 4 | starch | 24.55 |
| 5 | xanthan gum | 2.64 |
| 6 | banana flavour | 1.00 |
| 7 | silicon dioxide | 1.00 |
| 8 | talc | 2.12 |

| | |
|---|---|
| Total weight of composition | 263 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Methylphenidate HCl and sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating Solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3: (Functional Coating on Pre-Coated Active Ingredient-Resin Complex (Sustained Release Coating)—35% w/w Build Up

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4: (Preparation of Placebo Granules)

Placebo granules were prepared by mixing poloxamer 188, sucrose, sodium citrate, anhydrous citric acid, sucralose, sodium methyl paraben and sodium propyl paraben in granulator. Granules obtained were dried.

Step 5: (Preparation of Powder for Suspension)

Functional coated granules (35% w/w) and placebo granules were mixed with starch, xanthan gum, banana flavour, silicon dioxide and talc. The final blend was packed in suitable container.

### Example 5: Powder for Suspension

### Formula for Active Ingredient-Resin Complexation (by Entrapment Process)

| | | |
|---|---|---|
| S. No. | Ingredients | mg/ml |

Step 1 Active ingredient-ion exchange resin complexation

| | | |
|---|---|---|
| 1 | Methylphenidate HCl | 5.00 |
| 2 | sodium polystyrene sulfonate | 15.00 |
| 3 | purified water | Q.S |

| | |
|---|---|
| Total weight active ingredient-resin complex | 20.00 |

Step 2 Active ingredient-ion exchange resin entrapment in polymer

| | | |
|---|---|---|
| 4 | ethylcellulose | 4.00 |
| 5 | isopropyl alcohol | Q.S |
| 6 | purified water | Q.S |

| | |
|---|---|
| Total weight of entrapped active ingredient-resin complex | 24.00 |

Step 3 Solvation coating (pre-coating of active ingredient-resin complex),
buildup: 20% w/w

| | | |
|---|---|---|
| 7 | polyethylene glycol (PEG) | 4.80 |
| 8 | isopropyl alcohol and purified water | Q.S |

| | |
|---|---|
| Total weight of pre-coated active ingredient- resin complex | 28.8 |

Step 4a Functional coating on pre-coated active ingredient-resin complex
(sustained release 1 coating) - 35% w/w build up on 80 parts of
pre-coated active ingredient-ion exchange resin complex

| | | |
|---|---|---|
| 9 | ethylcellulose | 5.800 |
| 10 | hypromellose | 0.645 |
| 11 | dibutyl sebacate | 0.806 |
| 12 | talc | 0.806 |
| 13 | isopropyl alcohol and purified water | Q.S |

| | |
|---|---|
| Total weight functional coated granules (a) | 31.097 |

Step 4b Functional coating on pre-coated active ingredient-resin complex
(sustained release 2 coating) - 5% w/w build up on 20 parts of
Pre-coated active ingredient-ion exchange resin complex

| | | |
|---|---|---|
| 14 | ethylcellulose | 0.207 |
| 15 | hypromellose | 0.023 |
| 16 | dibutyl sebacate | 0.029 |
| 17 | talc | 0.029 |
| 18 | isopropyl alcohol and purified water | Q.S |

| | |
|---|---|
| Total weight functional coated granules b) | 6.048 |

### Formula for Placebo Granules

| S. No. | Ingredients | mg/ml |
|---|---|---|
| 1 | poloxamer 188 | 0.72 |
| 2 | sucrose | 200.10 |
| 3 | sodium citrate | 11.40 |
| 4 | anhydrous citric acid | 1.65 |
| 5 | sodium methyl paraben | 0.39 |
| 6 | sodium propyl paraben | 0.04 |
| 7 | sucralose | 1.15 |
| 8 | purified water | q.s |

| | |
|---|---|
| Total weight of placebo granules (c) | 215.5 |

### Formula for Powder for Oral ER Suspension

| S. No. | Ingredients | mg/ml |
|---|---|---|
| 1 | placebo granules (c) | 215.5 |
| 2 | functional coated granules (a) | 31.097 |
| 3 | functional coated granules (b) | 6.048 |
| 4 | starch | 24.55 |
| 5 | xanthan gum | 2.64 |
| 6 | banana flavour | 1.00 |
| 7 | silicon dioxide | 1.00 |
| 8 | talc | 2.12 |

| | |
|---|---|
| Total weight of composition | 283 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Methylphenidate HCl and Sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Active Ingredient-Ion Exchange Resin Complex Entrapment in Polymer)

Dissolve ethyl cellulose in Isopropyl cellulose under stirring. Add the dried active ingredient-ion exchange resin complex into the ethyl cellulose solution to get a uniform dispersion and keep under stirring for 1-2 hours. Add the dispersion slowly into a container containing purified water kept under constant stirring. Active ingredient-ion exchange resin complexes entrapped in polymer matrix shall be obtained. Collect the complexes and dry using a fluidized bed dryer or tray drier. Mill the complexes, if required.

Step 3: (Pre-Coating of Active Ingredient-Resin Complex Entrapped in Polymer)

The coating solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex entrapped in polymer.

Step 4a: (Functional Coating on Pre-Coated Active Ingredient-Resin Complex (Sustained Release 1 Coating)—50% w/w Build Up on 80 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 5: (Preparation of Placebo Granules)

Placebo granules were prepared by mixing poloxamer 188, sucrose, sodium citrate, anhydrous citric acid sucralose, sodium methyl paraben and sodium propyl paraben in granulator. Granules obtained were dried.

Step 6: (Preparation of Powder for Suspension)

Functional coated granules (50% w/w), functional coated granules (5% w/w) and placebo granules were mixed with starch, xanthan gum, banana flavour, silicon dioxide and talc. The final blend was packed in suitable container.

### Example 6: Ready to Use Suspension

### Formula for Active Ingredient-Ion Exchange Resin Complexation

| | | |
|---|---|---|
| S. No. | Ingredients | mg/5 ml |

Step 1 Active ingredient-ion exchange resin complexation

| | | |
|---|---|---|
| 1 | Dextromethorphan Hydrobromide | 30.00 |
| 2 | sodium polystyrene sulfonate | 75.00 |
| 3 | purified water | Q.S |

| | |
|---|---|
| Total weight of active ingredient- resin complex | 105.0 |

Step 2 Solvation coating (pre-coating of active ingredient-resin complex),
Buildup: 20% w/w

| | | |
|---|---|---|
| 4 | polyethylene glycol (PEG) | 21.00 |
| 5 | isopropyl alcohol and purified water | Q.S |

| | |
|---|---|
| Total weight of pre-coated active ingredient-resin complex | 126.00 |

Step 3a Functional coating on pre-coated active ingredient-resin complex
(sustained release 1 coating) - 40% w/w build up on 80 parts of
pre-coated active ingredient-ion exchange resin complex

| | | |
|---|---|---|
| 6 | ethylcellulose | 29.03 |
| 7 | hypromellose | 3.23 |
| 8 | dibutyl sebacate | 4.03 |
| 9 | talc | 4.03 |
| 10 | isopropyl alcohol and purified water | Q.S |

| | |
|---|---|
| Total weight functional coated granules (a) | 141.12 |

Step 3b Functional coating on pre-coated active ingredient-resin complex
(sustained release 2 coating) - 5% w/w build up on 20 parts of
pre-coated active ingredient-ion exchange resin complex

| | | |
|---|---|---|
| 11 | ethylcellulose | 0.91 |
| 12 | hypromellose | 0.10 |
| 13 | dibutyl sebacate | 0.13 |
| 14 | talc | 0.13 |
| 15 | isopropyl alcohol and purified water | Q.S |

| | |
|---|---|
| Total weight of functional coated granules b) | 26.47 |

### Formula for Oral Suspension

| S. No | Ingredients | mg/5 ml |
|---|---|---|
| 1 | functional coated granules (sustained release coated | 141.12 |
| | particulates a - 40% w/w) | |
| 2 | functional coated granules (sustained release coated | 26.47 |
| | particulates b - 5% w/w) | |
| 3 | sucrose | 1750 |
| 4 | methyl paraben | 5 |
| 5 | propyl paraben | 0.5 |
| 6 | sodium metabisulphite | 1.25 |
| 7 | glycerol anhydrous | 200 |
| 8 | xanthan gum | 10 |
| 9 | high fructose corn syrup | 650 |
| 10 | microcrystalline cellulose and carboxymethyl cellulose | 62.5 |
| 11 | FD&C no. 6 | 0.125 |
| 12 | flavour orange | 8.5 |
| 13 | polysorbate 80 | 10 |
| 14 | anhydrous citric acid | 1.5 |
| 15 | purified water | Q.S. |

| | |
|---|---|
| Total weight of oral ER suspension | ~2867 |

### Manufacturing Process

Step 1: (Active Ingredient-Ion Exchange Resin Complex Preparation)

The resin is activated by soaking in water for 1-2 hours. Dextromethorphan hydrobromide was dispersed in water and heated to 75° C. to solubilize the active ingredient. The active ingredient-ion exchange resin complex was prepared by adding sodium polystyrene sulfonate to Dextromethorphan hydrobromide solution kept under stirring and under heating for 3-5 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—40% w/w Build Up on 80 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 4: (Preparation of Suspension)

Functional coated granules (40% w/w) and Functional coated granules (5% w/w) were mixed with suspension base prepared using sucrose, methyl paraben, propyl paraben, sodium metabisulphite, glycerol anhydrous, xanthan gum, high fructose corn syrup, MCC and CMC (Avicel RC 591), FD&C No. 6, flavour orange, polysorbate 80, anhydrous citric acid. The suspension was packed in suitable container.

### Example 7: Ready to Use Suspension

### Formula for Active Ingredient-Ion Exchange Resin Complexation (by Entrapment Process)

| | | |
|---|---|---|
| S. No. | Ingredients | mg/5 ml |

Step 1 Active ingredient-ion exchange resin complexation

| | | |
|---|---|---|
| 1 | Dextromethorphan Hydrobromide | 30.00 |
| 2 | sodium polystyrene sulfonate | 75.00 |
| 3 | purified water | Q.S |

| | |
|---|---|
| Total weight of active ingredient- resin complexation | 105.0 |

Step 2 Active ingredient-ion exchange resin entrapment in polymer

| | | |
|---|---|---|
| 4 | ethylcellulose | 21.00 |
| 5 | isopropyl alcohol | Q.S |
| 6 | purified water | Q.S |

| | |
|---|---|
| Total weight of active entrapped complex | 126.0 |

Step 3 Solvation coating (pre-coating of active ingredient-resin complex),
Buildup: 20% w/w

| | | |
|---|---|---|
| 7 | polyethylene glycol (PEG) | 25.2 |
| 8 | isopropyl alcohol and purified water | Q.S |

| | |
|---|---|
| Total weight of pre-coated active ingredient- resin complex | 151.2 |

Step 4a Functional coating on pre-coated active ingredient-resin complex
(sustained release 1 coating) - 30% w/w build up on 80 parts of
pre-coated active ingredient-ion exchange resin complex

| | | |
|---|---|---|
| 9 | ethylcellulose | 26.13 |
| 10 | hypromellose | 2.90 |
| 11 | dibutyl sebacate | 3.66 |
| 12 | talc | 3.66 |
| 13 | isopropyl alcohol and purified water | Q.S |

| | |
|---|---|
| Total weight of functional coated granules (a) | 157.31 |

Step 4b Functional coating on pre-coated active ingredient-resin complex
(sustained release 2 coating) - 5% w/w build up on 20 parts of
pre-coated active ingredient-ion exchange resin complex

| | | |
|---|---|---|
| 14 | ethylcellulose | 1.09 |
| 15 | hypromellose | 0.12 |
| 16 | dibutyl sebacate | 0.15 |
| 17 | talc | 0.15 |
| 18 | isopropyl alcohol and purified water | Q.S |

| | |
|---|---|
| Total weight of functional coated granules b) | 31.75 |

### Formula for Oral Suspension

| S. No | Ingredients | mg/5 ml |
|---|---|---|
| 1 | functional coated granules (sustained release coated | 157.31 |
| | particulates a - 30% w/w) | |
| 2 | functional coated granules (sustained release coated | 31.75 |
| | particulates b - 5% w/w) | |
| 3 | sucrose | 1750 |
| 4 | methyl paraben | 5 |
| 5 | propyl paraben | 0.5 |
| 6 | sodium metabisulphite | 1.25 |
| 7 | glycerol anhydrous | 200 |
| 8 | xanthan gum | 10 |
| 9 | high fructose corn syrup | 650 |
| 10 | MCC and CMC (Avicel RC 591) | 62.5 |
| 11 | FD&C No. 6 | 0.125 |
| 12 | flavour orange | 8.5 |
| 13 | polysorbate 80 | 10 |
| 14 | anhydrous citric acid | 1.5 |
| 15 | Purified water | Q.S. |

| | |
|---|---|
| Total weight of oral ER suspension | ~2888 |

### Manufacturing Process

Step 1: (Active Ingredient-Ion Exchange Resin Complex Preparation)

The resin is activated by soaking in water for 1-2 hours. Dextromethorphan Hydrobromide was dispersed in water and heated to 75° C. to solubilize the active ingredient. The active ingredient-ion exchange resin complex was prepared by adding sodium polystyrene sulfonate to Dextromethorphan Hydrobromide solution kept under stirring and under heating for 3-5 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Active Ingredient-Ion Exchange Resin Complex Entrapment in Polymer)

Dissolve ethyl cellulose in isopropyl cellulose under stirring. Add the dried active ingredient-ion exchange resin complex into the ethyl cellulose solution to get a uniform dispersion and keep under stirring for 1-2 hours. Add the dispersion slowly into a container containing purified water kept under constant stirring. Active ingredient-ion exchange resin complexes entrapped in polymer matrix shall be obtained. Collect the complexes and dry using a fluidized bed dryer or tray drier. Mill the complexes, if required.

Step 3: (Pre-Coating of Active Ingredient-Resin Complex Entrapped in Polymer)

The coating solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex entrapped in polymer.

Step 4a: (Functional Coating on Pre-Coated Active Ingredient-Resin Complex (Sustained Release 1 Coating)—30% w/w Build Up on 80 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 4b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 5: (Preparation of Suspension)

Functional coated granules (30% w/w) and functional coated granules (5% w/w) were mixed with suspension base prepared using sucrose, methyl paraben, propyl paraben, sodium metabisulphite, glycerol anhydrous, xanthan gum, high fructose corn syrup, MCC and CMC (Avicel RC 591), FD&C No. 6, flavour orange, polysorbate 80, anhydrous citric acid. The suspension was packed in suitable container.

### Example 8: Ready to Use Suspension

### Formula for Active Ingredient-Ion Exchange Resin Complexation

| | | |
|---|---|---|
| S. No. | Ingredients | mg/5 ml |

Step 1 Active ingredient-ion exchange resin complexation

| | | |
|---|---|---|
| 1 | Dextromethorphan Hydrobromide | 30.00 |
| 2 | sodium polystyrene sulfonate | 75.00 |
| 3 | purified water | Q.S |

| | |
|---|---|
| Total weight of active ingredient- resin complex | 105.00 |

Step 2 Solvation coating (pre-coating of active ingredient-resin complex),
Buildup: 20% w/w

| | | |
|---|---|---|
| 4 | polyethylene glycol (PEG) | 20.8 |
| 5 | disodium EDTA | 0.20 |
| 6 | Purified water | Q.S |

| | |
|---|---|
| Total weight of pre-coated active ingredient-resin complex | 126.00 |

Step 3a Functional coating on pre-coated active ingredient-resin complex
(sustained release 1 coating) - 40% w/w build up on 80 parts of
pre-coated active ingredient-ion exchange resin complex

| | | |
|---|---|---|
| 7 | ethylcellulose | 29.03 |
| 8 | hypromellose | 3.23 |
| 9 | dibutyl sebacate | 4.03 |
| 10 | talc | 4.03 |
| 11 | isopropyl alcohol and purified water | Q.S |

| | |
|---|---|
| Total weight of functional coated granules (a) | 141.12 |

Step 3b Functional coating on pre-coated active ingredient-resin complex
(sustained release 2 coating) - 5% w/w build up on 20 parts of
pre-coated active ingredient-ion exchange resin complex

| | | |
|---|---|---|
| 12 | ethylcellulose | 0.91 |
| 13 | hypromellose | 0.10 |
| 14 | dibutyl sebacate | 0.13 |
| 15 | talc | 0.13 |
| 16 | isopropyl alcohol and purified water | Q.S |

| | |
|---|---|
| Total weight of functional coated granules b) | 26.47 |

### Formula for Oral Suspension

| S. No | Ingredients | mg/5 ml |
|---|---|---|
| 1 | functional coated granules (sustained release coated | 141.12 |
| | particulates a - 40% w/w) | |
| 2 | functional coated granules (sustained release coated | 26.47 |
| | particulates b - 5% w/w) | |
| 3 | sucrose | 1750 |
| 4 | methyl paraben | 5 |
| 5 | propyl paraben | 0.5 |
| 6 | glycerol anhydrous | 200 |
| 7 | xanthan gum | 10 |
| 8 | high fructose corn syrup | 650 |
| 9 | MCC and CMC (Avicel RC 591) | 62.5 |
| 10 | FD&C No. 6 | 0.125 |
| 11 | flavour orange | 8.5 |
| 12 | polysorbate 80 | 10 |
| 13 | anhydrous citric acid | 1.5 |
| 14 | purified water | Q.S. |

| | |
|---|---|
| Total weight of oral ER suspension | ~2866 |

### Manufacturing Process

Step 1: (Active Ingredient-Ion Exchange Resin Complex Preparation)

The resin is activated by soaking in water for 1-2 hours. Dextromethorphan Hydrobromide was dispersed in water and heated to 75° C. to solubilize the active ingredient. The active ingredient-ion exchange resin complex was prepared by adding sodium polystyrene sulfonate to Dextromethorphan Hydrobromide solution kept under stirring and under heating for 3-5 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating Solution was prepared by dispersing polyethylene glycol (PEG) in purified water and adding activated disodium EDTA under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—40% w/w Build Up on 80 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 4: (Preparation of Suspension)

Functional coated granules (40% w/w) and functional coated granules (5% w/w) were mixed with suspension base prepared using sucrose, methyl paraben, propyl paraben, glycerol anhydrous, xanthan gum, high fructose corn syrup, MCC and CMC (Avicel 3.0 RC 591), FD&C No. 6, flavour orange, polysorbate 80, anhydrous citric acid. The suspension was packed in suitable container.

### Example 9: Ready to Use Suspension

### Formula for Active Ingredient-Ion Exchange Resin Complexation

| | | |
|---|---|---|
| S. No. | Ingredients | mg/5 ml |

Step 1 Active ingredient-ion exchange resin complexation

| | | |
|---|---|---|
| 1 | Dextromethorphan Hydrobromide | 30.00 |
| 2 | sodium polystyrene sulfonate | 75.00 |
| 3 | purified water | Q.S |

| | |
|---|---|
| Total weight of pre-coated active ingredient- resin complex | 105.0 |

Step 2 Solvation coating (pre-coating of active ingredient-resin complex),
Buildup: 20% w/w

| | | |
|---|---|---|
| 4 | povidone | 21.00 |
| 5 | isopropyl alcohol and purified water | Q.S |

| | |
|---|---|
| Total weight of pre-coated active ingredient-resin complex | 126.00 |

Step 3a Functional coating on pre-coated active ingredient-resin complex
(sustained release 1 coating) - 40% w/w build up on 80 parts of
pre-coated active ingredient-ion exchange resin complex

| | | |
|---|---|---|
| 6 | ethylcellulose | 29.03 |
| 7 | hypromellose | 3.23 |
| 8 | dibutyl sebacate | 4.03 |
| 9 | talc | 4.03 |
| 10 | isopropyl alcohol and purified water | Q.S |

| | |
|---|---|
| Total weight of functional coated granules (a) | 141.12 |

Step 3b Functional coating on pre-coated active ingredient-resin complex
(sustained release 2 coating) - 5% w/w build up on 20 parts of
pre-coated active ingredient-ion exchange resin complex

| | | |
|---|---|---|
| 11 | ethylcellulose | 0.91 |
| 12 | hypromellose | 0.10 |
| 13 | dibutyl sebacate | 0.13 |
| 14 | talc | 0.13 |
| 15 | isopropyl alcohol and purified water | Q.S |

| | |
|---|---|
| Total weight functional coated granules b) | 26.47 |

### Formula for Oral Suspension

| S. No | Ingredients | mg/5 ml |
|---|---|---|
| 1 | functional coated granules (sustained release coated | 141.12 |
| | particulates a - 40% w/w) | |
| 2 | functional coated granules (sustained release coated | 26.47 |
| | particulates b - 5% w/w) | |
| 3 | sucrose | 1750 |
| 4 | methyl paraben | 5 |
| 5 | propyl paraben | 0.5 |
| 6 | sodium metabisulphite | 1.25 |
| 7 | glycerol anhydrous | 200 |
| 8 | xanthan gum | 10 |
| 9 | high fructose corn syrup | 650 |
| 10 | MCC and CMC (Avicel RC 591) | 62.5 |
| 11 | FD&C No. 6 | 0.125 |
| 12 | flavour orange | 8.5 |
| 13 | polysorbate 80 | 10 |
| 14 | anhydrous citric acid | 1.5 |
| 15 | purified water | Q.S. |

| | |
|---|---|
| Total weight of oral suspension | ~2867 |

### Manufacturing Process

Step 1: (Active Ingredient-Ion Exchange Resin Complex Preparation)

The resin is activated by soaking in water for 1-2 hours. Dextromethorphan Hydrobromide was dispersed in water and heated to 75° C. to solubilize the active ingredient. The active ingredient-ion exchange resin complex was prepared by adding sodium polystyrene sulfonate to Dextromethorphan Hydrobromide solution kept under stirring and under heating for 3-5 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating solution was prepared by dispersing povidone in isopropyl alcohol and purified water under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—40% w/w Build Up on 80 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, dibutyl sebacate, hypromellose and talc in isopropyl alcohol and purified water under stirring for 45 minutes. The coating process was performed in a fluid bed processor by applying coating solution to the pre-coated active ingredient-resin complex.

Step 4: (Preparation of Suspension)

Functional coated granules (40% w/w) and functional coated granules (5% w/w) were mixed with suspension base prepared using sucrose, methyl paraben, propyl paraben, sodium metabisulphite, glycerol anhydrous, xanthan gum, high fructose corn syrup, MCC and CMC (Avicel RC 591), FD&C No. 6, flavour orange, polysorbate 80, anhydrous citric acid. The suspension was packed in suitable container.

### Example 10: Powder for Suspension

### Formula for Active Ingredient-Ion Exchange Resin Complexation

| | | |
|---|---|---|
| S. No. | Ingredients | mg/ml |

Step 1 Active ingredient-ion exchange resin complexation

| | | |
|---|---|---|
| 1 | Metformin HCl | 100.00 |
| 2 | sodium polystyrene sulfonate | 300.00 |
| 3 | purified water | Q.S |

| | |
|---|---|
| Total weight of active ingredient- resin complex | 400.00 |

Step 2 Solvation coating (pre-coating of active ingredient-resin
complex), Buildup: 20% w/w

| | | |
|---|---|---|
| 4 | polyethylene glycol (PEG) | 80.00 |
| 5 | isopropyl alcohol and purified water | Q.S |

| | |
|---|---|
| Total weight of pre-coated active ingredient- resin complex | 480.00 |

Step 3a Functional coating on pre-coated active ingredient-resin complex
(sustained release 1 coating) - 50% w/w build up on 90 parts of
pre-coated active ingredient-ion exchange resin complex

| | | |
|---|---|---|
| 6 | ethylcellulose | 155.70 |
| 7 | hypromellose | 17.30 |
| 8 | dibutyl sebacate | 22.00 |
| 9 | talc | 22.00 |
| 10 | isopropyl alcohol and purified water | Q.S |

| | |
|---|---|
| Total weight of functional coated granules (a) | 649.00 |

Step 3b Functional coating on pre-coated active ingredient-resin complex
(sustained release 2 coating) - 5% w/w build up on 10 parts of
pre-coated active ingredient-ion exchange resin complex

| | | |
|---|---|---|
| 11 | ethylcellulose | 1.73 |
| 12 | hypromellose | 0.19 |
| 13 | dibutyl sebacate | 0.24 |
| 14 | talc | 0.24 |
| 15 | isopropyl alcohol and purified water | Q.S |

| | |
|---|---|
| Total weight of functional coated granules b) | 50.40 |

### Formula for Placebo Granules

| S. No. | Ingredients | mg/ml |
|---|---|---|
| 1 | microcrystalline cellulose - | 20.00 |
| | carboxymethyl cellulose sodium | |
| 2 | methyl paraben | 1.80 |
| 3 | propyl paraben | 0.20 |
| 4 | xanthan gum | 1.50 |
| 5 | strawberry flavor | 1.50 |
| 6 | xylitol | 450.00 |

| | |
|---|---|
| Total weight of placebo granules (c) | 475.0 |

### Formula for Powder for Oral Suspension

| S. No. | Ingredients | mg/ml |
|---|---|---|
| 1 | Placebo granules (c) | 475.00 |
| 2 | Functional coated granules (a) | 649.00 |
| 3 | Functional coated granules (b) | 50.40 |

| | |
|---|---|
| Total weight of composition | ~1174 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Metformin HCl and sodium polystyrene sulfonate (Purolite) to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—50% w/w Build Up on 90 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient Resin Complex (Sustained Release 2 Coating)—5% w/w Build Up on 10 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4: (Preparation of Placebo Granules)

Placebo granules were prepared by mixing microcrystalline cellulose-carboxymethyl cellulose sodium, methyl paraben, propyl paraben, xanthan gum, strawberry flavour, xylitol in a blender or alternatively granulated in a granulator. Granules obtained after granulation were dried.

Step 5: (Preparation of Powder for Suspension)

Functional coated granules (50% w/w), functional coated granules (5% w/w) and placebo granules were mixed. The final blend was packed in suitable container.

### Example 11: Powder for Suspension

### Formula for Active Ingredient-Ion Exchange Resin Complexation

| | | |
|---|---|---|
| S. No. | Ingredients | mg/ml |

Step 1 Active ingredient-ion exchange resin complexation

| | | |
|---|---|---|
| 1 | Galantamine Hydrobromide | 30.76 |
| 2 | sodium polystyrene sulfonate | 92.24 |
| 3 | purified water | Q.S |

| | |
|---|---|
| Total weight of active ingredient- resin complex | 123.00 |

Step 2 Solvation coating (pre-coating of active ingredient-resin complex),
Buildup: 20% w/w

| | | |
|---|---|---|
| 4 | polyethylene glycol (PEG) | 25.00 |
| 5 | isopropyl alcohol and purified water | Q.S |

| | |
|---|---|
| Total weight of pre-coated active ingredient-resin complex | 148.00 |

Step 3 Functional coating on pre-coated active ingredient-resin complex
(sustained release coating) - 40% w/w build up

| | | |
|---|---|---|
| 6 | ethylcellulose | 42.62 |
| 7 | hypromellose | 4.74 |
| 8 | dibutyl sebacate | 5.92 |
| 9 | talc | 5.92 |
| 10 | isopropyl alcohol and purified water | Q.S |

| | |
|---|---|
| Total weight of functional coated granules | 199.2 |

### Formula for Placebo Granules

| S. No. | Ingredients | mg/ml |
|---|---|---|
| 1 | microcrystalline cellulose - | 15.00 |
| | carboxymethyl cellulose sodium | |
| 2 | methyl paraben | 1.50 |
| 3 | propyl paraben | 0.15 |
| 4 | xanthan gum | 1.80 |
| 5 | strawberry flavor | 1.00 |
| 6 | xylitol | 475.55 |

| | |
|---|---|
| Total weight of placebo granules | 495.00 |

### Formula for Powder for Oral Suspension

| S. No. | Ingredients | mg/ml |
|---|---|---|
| 1 | placebo granules | 495.00 |
| 2 | functional coated granules | 199.2 |

| | |
|---|---|
| Total weight of composition | ~694 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Galantamine Hydrobromide and sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3: (Functional Coating on Pre-Coated Active Ingredient-Resin Complex (Sustained Release Coating)—40% w/w Build Up on Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4: (Preparation of Placebo Granules)

Placebo granules were prepared by mixing microcrystalline cellulose-carboxymethyl cellulose sodium, methyl paraben, propyl paraben, xanthan gum, strawberry flavour, xylitol in a blender or alternatively granulated in a granulator. Granules obtained after granulation were dried.

Step 5: (Preparation of Powder for Suspension)

Functional coated granules and placebo granules were mixed. The final blend was packed in suitable container.

### Example 12: Powder for Suspension

### Formula for Active Ingredient-Ion Exchange Resin Complexation

| | | |
|---|---|---|
| S. No. | Ingredients | mg/ml |

Step 1 Active ingredient-ion exchange resin complexation

| | | |
|---|---|---|
| 1 | Galantamine Hydrobromide | 30.76 |
| 2 | sodium polystyrene sulfonate | 92.24 |
| 3 | purified water | Q.S |

| | |
|---|---|
| Total weight of active ingredient- resin complex | 123.00 |

Step 2 Solvation coating (Pre-coating of active ingredient-resin
complex), Buildup: 20% w/w

| | | |
|---|---|---|
| 4 | polyethylene glycol (PEG) | 25.00 |
| 5 | isopropyl alcohol and purified water | Q.S |

| | |
|---|---|
| Total weight of pre-coated active ingredient-resin complex | 148.00 |

Step 3a Functional coating on pre-coated active ingredient-resin complex
(sustained release 1 coating) - 50% w/w build up on 80 parts of
pre-coated active ingredient-ion exchange resin complex

| | | |
|---|---|---|
| 6 | ethylcellulose | 42.62 |
| 7 | hypromellose | 4.74 |
| 8 | dibutyl sebacate | 5.92 |
| 9 | talc | 5.92 |
| 10 | isopropyl alcohol and purified water | Q.S |

| | |
|---|---|
| Total weight of functional coated granules (a) | 177.6 |

Step 3b Functional coating on pre-coated active ingredient-resin complex
(sustained release 2 coating) - 5% w/w build up on 20 parts of
pre-coated active ingredient-ion exchange resin complex

| | | |
|---|---|---|
| 11 | ethylcellulose | 1.07 |
| 12 | hypromellose | 0.12 |
| 13 | dibutyl sebacate | 0.15 |
| 14 | talc | 0.15 |
| 15 | isopropyl alcohol and purified water | Q.S |

| | |
|---|---|
| Total weight of functional coated granules b) | 31.09 |

### Formula for Placebo Granules

| S. No. | Ingredients | mg/ml |
|---|---|---|
| 1 | microcrystalline cellulose - | 15.00 |
| | carboxymethyl cellulose sodium | |
| 2 | methyl paraben | 1.50 |
| 3 | propyl paraben | 0.15 |
| 4 | xanthan gum | 1.80 |
| 5 | strawberry flavor | 1.00 |
| 6 | xylitol | 475.55 |

| | |
|---|---|
| Total weight of placebo granules (c) | 495.00 |

### Formula for Powder for Oral ER Suspension

| S. No. | Ingredients | mg/ml |
|---|---|---|
| 1 | placebo granules (c) | 495.00 |
| 2 | functional coated granules (a) | 177.6 |
| 3 | functional coated granules (b) | 31.09 |

| | |
|---|---|
| Total weight of composition | ~704 |

### Manufacturing Process

Step 1: (Active Ingredient-Ion Exchange Resin Complex Preparation)

The resin is activated by soaking in water for 1-2 hours. The active ingredient-ion exchange resin complex was prepared by adding Galantamine Hydrobromide and sodium polystyrene sulfonate to purified water and stirred for 7-8 hours. After complexation, the active ingredient-ion exchange resin dispersion was kept still for active ingredient-ion exchange resin complex to settle down and the solvent was drained. The wet drug-ion exchange resin complex was collected by filtration and dried using fluidized bed dryer.

Step 2: (Pre-Coating of Active Ingredient-Resin Complex)

The coating Solution was prepared by dispersing polyethylene glycol (PEG) in isopropyl alcohol and purified water under stirring for 45 minutes. The coating process was performed in a fluid bed processor by applying coating solution to the active ingredient-ion exchange resin complex.

Step 3a: (Functional Coating on Pre-Coated Active Ingredient-Resin Complex (Sustained Release 1 Coating)—50% w/w Build Up on 80 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 3b: Functional Coating on Pre-Coated Active Ingredient-Resin Complex (Sustained Release 2 Coating)—5% w/w Build Up on 20 Parts of Pre-Coated Active Ingredient-Ion Exchange Resin Complex

The coating solution was prepared by dispersing ethylcellulose, hypromellose, dibutyl sebacate, talc in isopropyl alcohol and purified water under stirring for 45 minutes. The coating process was performed in a fluid bed processor by applying coating solution to the pre-coated active ingredient-resin complex.

Step 4: (Preparation of Placebo Granules)

Placebo granules were prepared by mixing microcrystalline cellulose-carboxymethyl cellulose sodium, methyl paraben, propyl paraben, xanthan gum, strawberry flavour, xylitol in a blender or alternatively granulated in a granulator. Granules obtained after granulation were dried.

Step 5: (Preparation of Powder for Suspension)

Functional coated granules (50% w/w), functional coated granules (5% w/w) and placebo granules were mixed. The final blend was packed in suitable container.

Dissolution Data

The dissolution test is performed for the compositions of example 2, example 6 and example 10 which are given below in tables 1 to 3 respectively:

TABLE 1
Methylphenidate HCl Powder for suspension
0.4M Phosphate buffer, Paddle, 900 ml

| Time points (hr) | Test product (% release) |
|---|---|
| 0.25 | 25 |
| 0.5 | 35 |
| 1 | 47 |
| 2 | 65 |
| 3 | 72 |
| 4 | 86 |
| 6 | 90 |
| 8 | 95 |

TABLE 2
Dextromethorphan Hydrobromide suspension
0.1N HCl followed by 6.8 phosphate buffer,
900 ml, Paddle

| Time points (hr) | Test product (% release) |
|---|---|
| 1 | 19 |
| 1.5 | 47 |
| 2 | 55 |
| 2.5 | 59 |
| 3 | 65 |
| 4 | 68 |
| 6 | 77 |
| 8 | 85 |

TABLE 3
Metformin Hydrochloride powder for suspension
6.8 phosphate buffer, 1000 ml, Paddle

| Time points (hr) | Test product (% release) |
|---|---|
| 0.5 | 22 |
| 1 | 26 |
| 2 | 55 |
| 3 | 78 |
| 4 | 82 |
| 6 | 88 |
| 8 | 95 |

Stability Data

The above formulations were found to be stable at accelerated stability conditions for a period of 6 months. The stability data performed for the compositions of example 2, example 6 and example 10 is shown below in tables 4 to 6 respectively:

TABLE 4
Methylphenidate HCl Powder for suspension (after reconstitution)

| Sr. No. | Tests | | Initial | 6 month |
|---|---|---|---|---|
| 1 | Appearance | | Beige to tan | Beige to tan |
| | | | coloured | coloured |
| | | | suspension | suspension |
| 3 | Dissolution | 0.5 hr | 35 | 28 |
| | (%) | 3 hr | 72 | 69 |
| | | 8 hr | 95 | 91 |

| 4 | pH | 4.5 | 4.2 |
|---|---|---|---|
| 5 | Viscosity (cps) | 515 | 475 |
| 6 | Related Substances | | |
| | Methylphenidate | 0.3 | 1.0 |
| | related compound A | | |
| | Erythro isomer | 0.05 | 0.2 |
| | Any unspecified | 0.08 | 0.1 |
| | impurity (%) | | |
| | Total impurities (%) | 1.1 | 1.8 |
| 7 | Assay (%) | 97.5 | 98.7 |

TABLE 5
Dextromethorphan Hydrobromide suspension

| Sr. No. | Tests | | Initial | 6 month |
|---|---|---|---|---|
| 1 | Appearance | | Orange coloured | Orange coloured |
| | | | suspension | suspension |
| 3 | Dissolution | 1 hr | 19 | 22 |
| | (%) | 4 hr | 68 | 65 |
| | | 8 hr | 85 | 89 |

| 4 | pH | 4.2 | 4.6 |
|---|---|---|---|
| 5 | Viscosity (cps) | 275 | 250 |
| 6 | Related Substances | | |
| | Any unspecified | 0.05 | 0.1 |
| | impurity (%) | | |
| | Total impurities (%) | 0..9 | 0.8 |
| 7 | Assay (%) | 99.0 | 98.6 |

TABLE 6
Metformin Hydrochloride powder for
suspension (after reconstitution)

| Sr. No. | Tests | | Initial | 6 month |
|---|---|---|---|---|
| 1 | Appearance | | Beige to tan | Beige to tan |
| | | | coloured | coloured |
| | | | suspension | suspension |
| 3 | Dissolution | 0.5 hr | 22 | 25 |
| | (%) | 3 hr | 78 | 81 |
| | | 8 hr | 95 | 93 |

| 4 | pH | 5.5 | 5.7 |
|---|---|---|---|
| 5 | Viscosity (cps) | 605 | 595 |
| 6 | Related Substances | | |
| | Any unspecified | 0.05 | 0.07 |
| | impurity (%) | | |
| | Total impurities (%) | 0.90 | 1.02 |
| 7 | Assay (%) | 99.5 | 97.5 |

1. An extended-release suspension composition comprising active ingredient, activated resin and pharmaceutically acceptable excipients, wherein said composition is devoid of uncoated active ingredient-ion exchange resin complex portion and polyvinyl acetate.

2. The extended-release suspension composition as claimed in claim 1, wherein said composition comprising one or more functional barrier coatings comprising hypromellose and/or talc on active ingredient-ion exchange resin complex.

3. The extended-release suspension composition as claimed in claim 1, wherein said composition comprising;
a) polymeric coated active ingredient-ion exchange resin complex comprising:
(i) active ingredient particulate matrix comprising active ingredient-ion exchange resin complex,
(ii) solvation coating on the active ingredient-ion exchange resin complex,
(iii) one or more functional barrier coating on the pre-coated active ingredient—ion exchange resin complex obtained in step (ii), wherein said functional barrier coating provides modified release profile to said pharmaceutically acceptable active ingredient, and
b) pharmaceutically acceptable excipients.

4. The extended-release suspension composition as claimed in claim 1, wherein said composition comprising;
a) polymeric coated active ingredient-ion exchange resin complex comprising:
(i) active ingredient particulate matrix comprising active ingredient-ion exchange resin complex,
(ii) solvation coating on the active ingredient-ion exchange resin complex,
(iii) dual release functional barrier coatings on the pre-coated active ingredient-ion exchange resin complex obtained in step (ii), wherein said functional barrier coatings provides dual modified release profile to said pharmaceutically acceptable active ingredient, and
b) pharmaceutically acceptable excipients.

5. The extended-release suspension composition as claimed in claim 1, wherein said composition comprising;
a) polymeric coated active ingredient-ion exchange resin complex comprising:
(i) active ingredient particulate matrix comprising active ingredient-ion exchange resin complex,
(ii) entrapment of active ingredient-ion exchange resin complex in polymer,
(iii) solvation coating on the active ingredient-ion exchange resin complex obtained in step (ii),
(iv) dual release functional barrier coatings on the pre-coated active ingredient-ion exchange resin complex obtained in step (iii), wherein said functional barrier coatings provides dual modified release profile to said pharmaceutically acceptable active ingredient, and
b) pharmaceutically acceptable excipients.

6. The extended-release suspension composition as claimed in claim 1, wherein said composition comprising;
a) polymeric coated active ingredient-ion exchange resin complex comprising:
(i) active ingredient particulate matrix comprising active ingredient-sodium polystyrene sulfonate complex,
(ii) solvation coating on the active ingredient-sodium polystyrene sulfonate complex comprising polymers selected from polyethylene glycol, hypromellose and povidone,
(iii) one or more functional barrier coating on the pre-coated active ingredient-sodium polystyrene sulfonate complex obtained in step (ii), wherein said functional barrier coating comprising hypromellose and talc which provides modified release profile to said pharmaceutically acceptable active ingredient, and
b) pharmaceutically acceptable excipients.

7. The extended-release suspension composition as claimed in claim 1, wherein said composition comprising;
a) polymeric coated active ingredient-ion exchange resin complex comprising:
(i) active ingredient particulate matrix comprising active ingredient-sodium polystyrene sulfonate complex,
(ii) solvation coating on the active ingredient—sodium polystyrene sulfonate complex comprising polymers selected from polyethylene glycol, hypromellose and povidone,
(iii) dual release functional barrier coatings on the pre-coated active ingredient-sodium polystyrene sulfonate complex obtained in step (ii), wherein said functional barrier coatings comprising hypromellose and talc which provides dual modified release profile to said pharmaceutically acceptable active ingredient, and
b) pharmaceutically acceptable excipients.

8. The extended-release suspension composition as claimed in claim 1, wherein said composition comprising;
a) polymeric coated active ingredient-ion exchange resin complex comprising:
(i) active ingredient particulate matrix comprising active ingredient-sodium polystyrene sulfonate complex,
(ii) entrapment of active ingredient—sodium polystyrene sulfonate complex in ethylcellulose polymer,
(iii) solvation coating on the active ingredient-sodium polystyrene sulfonate complex comprising polymers selected from polyethylene glycol, hypromellose and povidone,
(iv) dual release functional barrier coatings on the pre-coated active ingredient-sodium polystyrene sulfonate complex obtained in step (ii), wherein said functional barrier coatings comprising hypromellose and talc which provides dual modified release profile to said pharmaceutically acceptable active ingredient, and
b) pharmaceutically acceptable excipients.

9. The extended-release suspension composition as claimed in claim 1, wherein said composition comprising functional coated active ingredient-ion exchange resin complex, placebo granules, and pharmaceutically acceptable excipients.

10. A process for the preparation of extended-release suspension composition, wherein said process comprises:
(a) preparing active ingredient-ion exchange resin complex,
(b) coating on active ingredient-ion exchange resin complex with solvation coating polymers,
(c) coating on pre-coated active ingredient-ion exchange resin complex with functional barrier coating polymers, and
(d) mixing of functional coated active ingredient-ion exchange resin complex with excipients.

11. The process for the preparation of extended-release suspension composition as claimed in claim 10, wherein said comprises;
(a) preparing active ingredient-ion exchange resin complex,
(b) coating on active ingredient-ion exchange resin complex with solvation coating polymers,
(c) coating on pre-coated active ingredient-ion exchange resin complex with functional barrier coating polymers,
(d) preparing the placebo granules with excipients,
(e) mixing of functional coated active ingredient-ion exchange resin complex particles obtained in steps (c) and placebo granules obtained in step (d) with pharmaceutically acceptable excipients, and
(f) packing the composition obtained in step (e) in suitable container.

12. The process for the preparation of extended-release suspension composition as claimed in claim 10, wherein said comprises;
(a) preparing active ingredient—ion exchange resin complex,
(b) coating on active ingredient-ion exchange resin complex with solvation coating polymers,
(c) coating 70% to 95% of pre-coated active ingredient-ion exchange resin complex particles obtained in step (b) with functional barrier coating polymers from 25% w/w to 60% w/w build up,
(d) coating 5% to 30% of pre-coated active ingredient-ion exchange resin complex particles obtained in step (b) with functional barrier coating polymers from 5% w/w to 15% w/w build up,
(e) mixing of functional coated active ingredient-ion exchange resin complex particles obtained in steps (c) and step (d) with pharmaceutically acceptable excipients, and
(f) packing the composition obtained in step (e) in suitable container.

13. The process for the preparation of extended-release suspension composition as claimed in claim 10, wherein said comprises;
(a) preparing active ingredient—ion exchange resin complex,
(b) coating on active ingredient-ion exchange resin complex with solvation coating polymers,
(c) entrapping the active ingredient-ion exchange resin complex obtained in step (b) with polymers,
(d) coating 70% to 95% of pre-coated active ingredient-ion exchange resin complex particles obtained in step (c) with functional barrier coating polymers from 25% w/w to 60% w/w build up,
(e) coating 5% to 30% of pre-coated active ingredient-ion exchange resin complex particles obtained in step (c) with functional barrier coating polymers from 5% w/w to 15% w/w build up,
(f) mixing of functional coated active ingredient-ion exchange resin complex particles obtained in steps (d) and step (e) with pharmaceutically acceptable excipients, and
(g) packing the composition obtained in step (f) in suitable container.

14. The process for the preparation of extended-release suspension composition as claimed in claim 10, wherein said comprises;
(a) preparing active ingredient-ion exchange resin complex by adding active ingredient and activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating pre-coated active ingredient-ion exchange resin complex particles obtained in step (b) with functional barrier coating solution comprising hypromellose and talc,
(d) mixing of functional coated active ingredient-ion exchange resin complex particles obtained in steps (c) with pharmaceutically acceptable excipients, and
(e) packing the composition obtained in step (d) in suitable container.

15. The process for the preparation of extended-release suspension composition as claimed in claim 10, wherein said comprises;
(a) preparing active ingredient-ion exchange resin complex by adding active ingredient and activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating pre-coated active ingredient-ion exchange resin complex particles obtained in step (b) with functional barrier coating solution comprising hypromellose and talc,
(d) preparing placebo granules with excipients,
(e) mixing of functional coated active ingredient-ion exchange resin complex particles obtained in steps (c) with placebo granules obtained in step (d) and pharmaceutically acceptable excipients, and
(f) packing the composition obtained in step (e) in suitable container.

16. The process for the preparation of extended-release suspension composition as claimed in claim 10, wherein said comprises;
(a) preparing active ingredient-ion exchange resin complex by adding active ingredient, activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating 70% to 95% of pre-coated active ingredient-ion exchange resin complex particles obtained in step (c) with functional barrier coating solution comprising hypromellose and talc from 25% w/w to 60% w/w build up,
(d) coating 5% to 30% of pre-coated active ingredient-ion exchange resin complex particles obtained in step (c) with functional barrier coating solution comprising hypromellose and talc from 5% w/w to 15% w/w build up,
(e) mixing of functional coated active ingredient-resin complex particles obtained in steps (c) and (d) with pharmaceutically acceptable excipients, and
(f) packing the composition obtained in step (e) in suitable container.

17. The process for the preparation of extended-release suspension composition as claimed in claim 10, wherein said comprises;
(a) preparing active ingredient-ion exchange resin complex by adding active ingredient, activated resin to solvent under stirring for 7-8 hours followed by drying,
(b) coating the active ingredient-ion exchange resin complex obtained in step (a) with solvation coating solution comprising polymer selected from polyethylene glycol, hypromellose and povidone,
(c) coating 70% to 95% of pre-coated active ingredient-ion exchange resin complex particles obtained in step (c) with functional barrier coating solution comprising hypromellose and talc from 25% w/w to 60% w/w build up,
(d) coating 5% to 30% of pre-coated active ingredient-ion exchange resin complex particles obtained in step (c) with functional barrier coating solution comprising hypromellose and talc from 5% w/w to 15% w/w build up,
(e) preparing placebo granules with excipients,
(f) mixing of functional coated active ingredient-resin complex particles obtained in steps (c) and (d) with placebo granules obtained in step (e) and pharmaceutically acceptable excipients, and
(g) packing the composition obtained in step (f) in suitable container.

18. The process for the preparation of extended-release suspension composition as claimed in claim 10, wherein said comprises;
(a) preparing active ingredient-ion exchange resin complex by adding active ingredient, activated resin to solvent under stirring for 7-8 hours followed by drying,
(b) entrapping the active ingredient-ion exchange resin complex obtained in step (a) using ethylcellulose solution,
(c) coating the active ingredient-ion exchange resin complex obtained in step (b) with solvation coating solution comprising polymer selected from polyethylene glycol, hypromellose and povidone,
(d) coating 70% to 95% of pre-coated active ingredient-ion exchange resin complex particles obtained in step (d) with functional barrier coating solution comprising hypromellose and talc from 25% w/w to 60% w/w build up,
(e) coating 5% to 30% of pre-coated active ingredient-ion exchange resin complex particles obtained in step (d) with functional barrier coating solution comprising hypromellose and talc from 5% w/w to 15% w/w build up,
(f) mixing of functional coated active ingredient-resin complex particles obtained in steps (d) and (e) with pharmaceutically acceptable excipients, and
(g) packing the composition obtained in step (f) in suitable container.

19. The process for the preparation of extended-release suspension composition as claimed in claim 10, wherein said comprises;
(a) preparing active ingredient-ion exchange resin complex by adding active ingredient, activated resin to solvent under stirring for 7-8 hours followed by drying,
(b) entrapping the active ingredient-ion exchange resin complex obtained in step (a) using ethylcellulose solution,
(c) coating the active ingredient-ion exchange resin complex obtained in step (b) with solvation coating solution comprising polymer selected from polyethylene glycol, hypromellose and povidone,
(d) coating 70% to 95% of pre-coated active ingredient-ion exchange resin complex particles obtained in step (d) with functional barrier coating solution comprising hypromellose and talc from 25% w/w to 60% w/w build up,
(e) coating 5% to 30% of pre-coated active ingredient-ion exchange resin complex particles obtained in step (d) with functional barrier coating solution comprising hypromellose and talc from 5% w/w to 15% w/w build up,
(f) preparing the placebo granules with excipients,
(g) mixing of functional coated active ingredient-resin complex particles obtained in steps (d) and (e) with placebo granules obtained in step (f) and pharmaceutically acceptable excipients, and
(h) packing the composition obtained in step (g) in suitable container.

20. The process for the preparation of extended-release suspension composition as claimed in claim 10, wherein said process comprises packing the functional coated active ingredient-ion exchange resin complex particles with pharmaceutically acceptable excipients and optionally with placebo granules in a suitable container.

21. The extended-release suspension composition as claimed in claim 1, wherein said active ingredient is selected from Metformin Hydrochloride, Galantamine Hydrobromide, Methylphenidate Hydrochloride, Amphetamine salts and Dextromethorphan Hydrobromide.

22. The extended-release suspension composition as claimed in claim 1, wherein said pharmaceutically acceptable excipients are ion exchange resin, chelating agent, solvation coating agent, entrapment polymers, more functional barrier coating polymers, plasticizer, taste masking agents, sweetening agent, buffering agents, anti-tacking agents, suspending agents/thickeners, preservatives, flavouring agents, surfactants, and lubricants.

23. The extended-release suspension composition as claimed in claim 22, wherein said ion exchange resin is sodium polystyrene sulfonate.

24. The extended-release suspension composition as claimed in claim 22, wherein said chelating agent as used herein is selected from EDTA, a salt of EDTA, diaminocyclohexanetetraacetic acid (DCTA), diethylenetriaminepentaacetic acid, bis(aminoethyl)glycolether-N,N,N′,N′-tetraacetic acid, iminodiacetic acid or a salt thereof.

25. The extended-release suspension composition as claimed in claim 22, wherein said solvation coating agent is selected from polyethylene glycol, mannitol, hypromellose, hydroxy propyl cellulose, povidone and methylcellulose.

26. The extended-release suspension composition as claimed in claim 22, wherein said entrapment polymers as used in the present invention are selected from ethyl cellulose, methyl cellulose, carboxymethyl cellulose, hydroxypropylmethyl cellulose, Methacrylic acid polymers & its co-polymers, sodium alginates, sodium carboxymethyl cellulose. Preferably, entrapment polymer is ethyl cellulose.

27. The extended-release suspension composition as claimed in claim 22, wherein said functional barrier coating polymers selected from the alkyl celluloses and derivatives thereof such as methylcellulose, ethylcellulose, hydroxyalkyl celluloses and derivatives thereof such as hydroxyethyl celluloses, hydroxypropyl celluloses, hydroxyalkyl alkylcelluloses and derivatives thereof such as the hydroxypropylmethyl celluloses (hypromelloses), carboxyalkylcelluloses such as carboxymethylcelluloses, polyvinylpyrrolidones, copolymers of N-vinylpyrrolidone and vinyl acetate or combinations of said polymers and derivatives and mixtures thereof.

28. The extended-release suspension composition as claimed in claim 22, wherein said plasticizer is selected from dibutyl sebacate, propylene glycol, polyethylene glycol, polyvinyl alcohol, triethyl citrate, acetyl triethyl citrate, acetyl tributyl citrate, tributyl citrate, triacetin, and Soluphor® P (2-pyrrolidone), and mixtures thereof.

29. The extended-release suspension composition as claimed in claim 22, wherein said taste masking agents are selected from potassium chloride, calcium chloride, sodium lactate, sodium chloride, dextrose, mannitol, sucrose, trehalose, and phosphate buffered saline, vanillin, ethyl vanillin, menthol, citric acid, fumaric acid ethyl maltitol, and tartaric acid, pullulan, hydroxypropylmethyl cellulose, hydroxyethyl cellulose, hydroxypropyl cellulose, polyvinyl pyrrolidone, carboxymethyl cellulose, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

30. The extended-release suspension composition as claimed in claim 22, wherein said sweetening agent is selected from sucrose, sucralose, xylitol, high fructose corn syrup and mixtures thereof.

31. The extended-release suspension composition as claimed in claim 22, wherein said pH adjusting agent is selected from citric acid, sodium citrate anhydrous, sodium citrate dihydrate and mixtures thereof.

32. The extended-release suspension composition as claimed in claim 22, wherein said anti-tacking agents are selected from silicon dioxide, titanium dioxide, alumina, talc, kaolin, powdered cellulose and microcrystalline cellulose and mixtures thereof.

33. The extended-release suspension composition as claimed in claim 22, wherein said suspending agents/thickeners are selected from xanthan gum, microcrystalline cellulose, starch, carboxymethyl cellulose, sodium carboxymethyl cellulose and mixtures thereof.

34. The extended-release suspension composition as claimed in claim 22, wherein said preservatives are selected from methylparaben, propylparaben, sodium methylparaben, sodium propylparaben, Sodium metabisulphite and mixtures thereof.

35. The extended-release suspension composition as claimed in claim 22, wherein said flavouring agent is selected from strawberry flavour, banana flavour, flavour orange, bubblegum flavour and mixtures thereof.

36. The extended-release suspension composition as claimed in claim 22, wherein said surfactants are selected from poloxamers (e.g., poloxamer 188), glycerol Anhydrous, polysorbate and mixtures thereof.

37. The extended-release suspension composition as claimed in claim 22, wherein said lubricants are selected from magnesium stearate, stearic acid, sodium stearyl fumarate, microcrystalline cellulose, crosslinked sodium carboxymethylcellulose, silica, aerosil, corn starch, sucrose fatty acid esters, polyethylene glycol, talc, stearic acid, calcium stearate and mixtures thereof.
